(19) United States
(12) Patent Application Publication
(10) Pub. No.: US 2023/0226556 A1
(43) Pub. Date: Jul. 20, 2023

(54) AIR PURIFICATION DEVICE AND AIR CONDITIONING DEVICE

(71) Applicant: MITSUBISHI HEAVY INDUSTRIES POWER ENVIRONMENTAL SOLUTIONS, LTD., Yokohama-Shi, Kanagawa (JP)

(72) Inventors: Kazutaka Tomimatsu, Hyogo (JP); Masaya Kato, Kanagawa (JP); Yasutoshi Ueda, Tokyo (JP)

(73) Assignee: MITSUBISHI HEAVY INDUSTRIES POWER ENVIRONMENTAL SOLUTIONS, LTD., Yokohama-Shi, Kanagawa (JP)

(21) Appl. No.: 17/928,710

(22) PCT Filed: Apr. 12, 2021
(86) PCT No.: PCT/JP2021/015129

§ 371 Date: Nov. 30, 2022

(30) Foreign Application Priority Data
Jul. 13, 2020 (JP) 2020-119957

(51) Int. Cl. B03C 3/017; F24F 8/192; B03C 3/41; B03C 3/47; B03C 3/019; B03C 3/155; B03C 3/68; B01D 53/00; B01D 53/86; B01D 53/88; A61L 9/015

(52) U.S. Cl. CPC B03C 3/017; F24F 8/192; B03C 3/41; B03C 3/47; B03C 3/019; B03C 3/155; B03C 3/68; B01D 53/007; B01D 53/8675; B01D 53/8696; B01D 53/885; A61L 9/015; B01D 2255/2073; B01D 2255/20753; B01D 2257/106; B01D 2259/804; B01D 2259/818; B01D 2255/9155; A61L 2209/14; A61L 2209/16; A61L 2209/11

(57) ABSTRACT

An air purification device including a flow passage through which air circulates; an electrical precipitator unit that is disposed in the flow passage and that includes a discharge electrode having a body unit and a corona discharge unit for corona discharge which protrudes from the body unit, and a collecting electrode disposed opposing the discharge electrode; an ozone removal unit that is disposed in the flow passage, and that is capable of removing ozone included in the circulating air, and a control unit that switches between a first mode in which air from which ozone has been removed is supplied from a downstream section of the flow passage to the outside, and a second mode in which air including ozone is supplied from the downstream section of the flow passage to the outside.

### TECHNICAL FIELD

The present disclosure relates to an air purification device and an air conditioning device.

### BACKGROUND ART

In air conditioning devices, HEPA filters are not normally used and medium-efficiency particulate air filters, compact electrostatic filters, and the like are installed as dust removal devices for particle collection. In performing air conditioning while recirculating indoor air under environmental conditions where there is little fine particulate matter such as PM2.5, the above configuration satisfies the environmental conditions.

In the case of indoor air recirculation by an air conditioning system using an air conditioning device, fine particulate matter (submicron particle) generated indoors, a virus emitted from a person, or the like is hardly collected by a filter installed in the air conditioning device due to the small particle size thereof. As a result, these substances continue to remain indoors.

PTL 1 discloses a technique in which the inside of the main body of the indoor unit of an air conditioner is provided with an ozone-ion generator for ozone and ionic wind generation by discharge so that the inside of the main body is purified and sterilization is performed by ozone diffusion.

### CITATION LIST

### Patent Literature

[PTL 1] International Publication No. 2012/035757

### SUMMARY OF INVENTION

### Technical Problem

When a HEPA filter is applied as an air conditioning device filter, energy consumption increases due to the pressure loss thereof. In an air conditioning device that takes in outside air, such as an air handling unit, HEPA filter application is disadvantageous in terms of energy. Accordingly, in an air conditioning device that treats a large amount of air using a medium-efficiency particulate air filter instead of a HEPA filter, fine particulate matter such as PM2.5 cannot be sufficiently removed, and thus an air purification device has to be separately installed in a case where a highly air-purified environment is required.

An air purification device may be provided with an electrical precipitator unit, and the electrical precipitator unit has a discharge electrode charging particles and a collecting electrode installed so as to face the discharge electrode. When the voltage that is applied to the discharge electrode of the electrical precipitator unit is increased, the amount of charge on submicron particles increases and an increase in electric field strength occurs to result in collection efficiency improvement. However, when the voltage applied to the discharge electrode of the electrical precipitator unit increases, the amount of ozone generated by corona discharge at the discharge electrode increases. Although it is desirable to improve the collection efficiency by increasing the voltage applied to the discharge electrode, an increase in ozone concentration adversely affects the human body, and thus the ozone concentration needs to be equal to or less than the environmental standard value while a person stays in a space when the electrical precipitator unit is used for air purification.

Ozone has the effect of inactivating viruses and sterilizing fungi adhering to walls, fittings, or furniture in spaces and coming into contact with air surfaces. However, when ozone generated by corona discharge at the discharge electrode is removed, the virus inactivation and fungus sterilization effect of ozone cannot be obtained.

The present disclosure has been made in view of such circumstances, and an object of the present disclosure is to provide an air purification device and an air conditioning device with which it is possible to improve the efficiency of collecting particularly fine particles without increasing pressure loss and deodorize or sterilize air in a space.

### Solution to Problem

In order to solve the above problems, the air purification device and the air conditioning device of the present disclosure adopt the following means.

In other words, an air purification device of the present disclosure includes: a flow path through which air flows; an electrical precipitator unit including a discharge electrode having a main body portion and a corona discharge portion for corona discharge protruding from the main body portion and a collecting electrode installed to face the discharge electrode and installed in the flow path; an ozone removal unit installed in the flow path and capable of removing ozone contained in the flowing air; and a first control unit switching between a first mode in which air from which the ozone is removed is supplied to an outside from a downstream portion of the flow path and a second mode in which air containing the ozone is supplied to the outside from the downstream portion of the flow path.

An air conditioning device according to the present disclosure includes: the air purification device described above; and an air conditioning unit, in which the air conditioning unit supplies air relatively high in temperature to a space and then supplies air relatively small in air amount and relatively low in temperature to the space, and the first control unit stops removal of the ozone by the ozone removal unit.

### Advantageous Effects of Invention

According to the present disclosure, it is possible to improve the efficiency of collecting particularly fine particles without increasing pressure loss and deodorize or sterilize air in a space.

### BRIEF DESCRIPTION OF DRAWINGS

FIG. 1 is a configuration diagram illustrating an air conditioning device according to a first embodiment of the present disclosure.

FIG. 2 is a configuration diagram illustrating a first example of an air handling unit of the air conditioning device according to the first embodiment of the present disclosure.

FIG. 3 is a configuration diagram illustrating the first example of the air handling unit of the air conditioning device according to the first embodiment of the present disclosure.

FIG. 4 is a configuration diagram illustrating a second example of the air handling unit of the air conditioning device according to the first embodiment of the present disclosure.

FIG. 5 is a configuration diagram illustrating a third example of the air handling unit of the air conditioning device according to the first embodiment of the present disclosure.

FIG. 6 is a configuration diagram illustrating the third example of the air handling unit of the air conditioning device according to the first embodiment of the present disclosure.

FIG. 7 is a lateral cross-sectional view illustrating an electrical precipitator unit and a medium-efficiency particulate air filter unit of the air handling unit according to the first embodiment of the present disclosure.

FIG. 8 is a vertical cross-sectional view illustrating an electrical precipitator unit of an air handling unit according to an embodiment of the present disclosure.

FIG. 9 is a perspective view illustrating the electrical precipitator unit of the air handling unit according to the first embodiment of the present disclosure.

FIG. 10 is a configuration diagram illustrating a modification example of the first example of the air handling unit of the air conditioning device according to the first embodiment of the present disclosure.

FIG. 11 is a configuration diagram illustrating a modification example of a third example of the air handling unit of the air conditioning device according to the first embodiment of the present disclosure.

FIG. 12 is a timing chart illustrating an example of the operation of the air handling unit of the air conditioning device according to the first embodiment of the present disclosure.

FIG. 13 is a timing chart illustrating another example of the operation of the air handling unit of the air conditioning device according to the first embodiment of the present disclosure.

FIG. 14 is a timing chart illustrating an example of the operation of a continuous energization condition of the air conditioning device according to the first embodiment of the present disclosure.

FIG. 15 is a timing chart illustrating an example of the operation of an intermittent energization condition of the air conditioning device according to the first embodiment of the present disclosure.

FIG. 16 is a graph illustrating the relationship between ozone concentration and electric power in the air conditioning device according to the first embodiment of the present disclosure.

FIG. 17 is a configuration diagram illustrating an air purification device according to a second embodiment of the present disclosure.

FIG. 18 is a timing chart illustrating an example of the operation of the air conditioning device according to the first embodiment of the present disclosure or the air purification device according to the second embodiment of the present disclosure.

FIG. 19 is a configuration diagram illustrating an air purification device according to a third embodiment of the present disclosure.

FIG. 20 is a configuration diagram illustrating the air purification device according to the third embodiment of the present disclosure.

FIG. 21 is a timing chart illustrating an example of the operation of the air purification device according to the third embodiment of the present disclosure.

### DESCRIPTION OF EMBODIMENTS

Hereinafter, an air conditioning device 1 according to an embodiment of the present disclosure will be described with reference to the drawings.

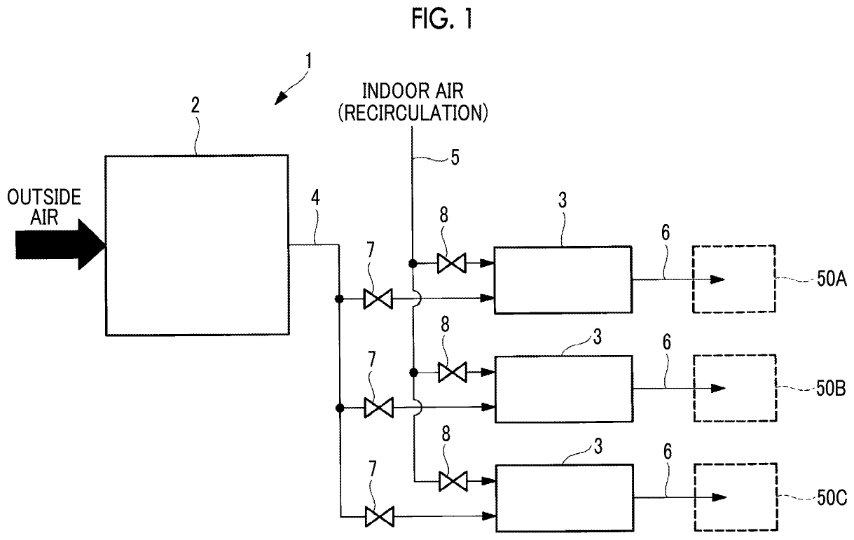

The air conditioning device 1 according to the present embodiment takes in external air (outside air) such as the atmosphere, performs temperature or humidity adjustment, and supplies the adjusted air to each space 50 provided in a building. As illustrated in FIG. 1, the air conditioning device 1 includes an outside air treatment air conditioner (hereinafter, referred to as “external air conditioner”) 2, a plurality of air handling units (hereinafter, referred to as “AHUs”) 3, ducts 4, 5, and 6, dampers 7 and 8, and so on.

The duct 4 is installed between the external air conditioner 2 and the AHU 3, one end thereof is connected to the external air conditioner 2, and the other end thereof is connected to the outside air intake port of the AHU 3. The duct 5 is installed between each space 50 (50A, 50B, and 50C in the example illustrated in FIG. 1) such as a room of a building or the like and the AHU 3, one end thereof is connected to the suction port provided in each of the spaces 50A, 50B, and 50C, and the other end thereof is connected to the recirculation air intake port of the AHU 3. The duct 6 is installed between the AHU 3 and each of the spaces 50A, 50B, and 50C, one end thereof is connected to the air discharge port of the AHU 3, and the other end thereof is connected to the outlet provided in each of the spaces 50A, 50B, and 50C.

The external air conditioner 2 takes in outside air, performs temperature and/or humidity adjustment of the outside air, and supplies the adjusted air to the AHU 3 via the duct 4. A filter, a heat exchanger, a humidifier, and so on are installed inside the casing of the external air conditioner 2.

The air supplied from the external air conditioner 2 to the plurality of AHUs 3 is branched by the duct 4 and supplied to each AHU 3. As for the duct 4, the damper 7 is installed upstream of the outside air intake port of each AHU 3, and the damper 7 adjusts the amount of outside air supplied to the AHU 3.

The AHU 3 takes in the air supplied from the external air conditioner 2 and the air from the space 50, performs temperature and/or humidity adjustment of the intake air, and supplies the adjusted air to the space 50 via the duct 6. An electrical precipitator unit 10, a filter unit 12, an air conditioning unit 13, and so on are installed inside a casing 9 of the AHU 3.

The air is taken into the AHU 3 from the space 50, and the AHU 3 readjusts the temperature and/or humidity of the air. In this manner, in the AHU 3, not only the outside air is taken in and supplied to the space 50, but also the air from the space 50 is recirculated, and energy efficiency can be enhanced as a result. As for the duct 5, the damper 8 is installed upstream of the recirculation air intake port of each AHU 3, and the damper 8 adjusts the amount of recirculation air supplied to the AHU 3.

In the air conditioning device 1 according to the present embodiment, first, the external air conditioner 2 takes in outside air, the external air conditioner 2 performs temperature and/or humidity adjustment of the outside air, and the air adjusted by the external air conditioner 2 is supplied to the AHU 3. Then, the AHU 3 takes in the air supplied from the external air conditioner 2 and the air from the space 50, the AHU 3 performs temperature and/or humidity adjustment of the intake air, and the air adjusted by the AHU 3 is supplied to the space 50. At this time, the amount of outside air taken into the AHU 3 from the external air conditioner 2 and the amount of recirculation air taken into the AHU 3 from the space 50 are adjusted by the dampers 7 and 8, respectively.

For example, in a cooling or heating season, the amount of outside air taken into the space 50 as fresh air is set to a relatively low ratio (for example, 30%) with respect to the total intake air amount. In the case of air conditioning device designed with energy saving taken into account, 100% of outside air may be taken in an intermediate season (spring or autumn), and adjustment is performed such that energy efficiency is enhanced by outside air intake being performed in accordance with the season.

Next, the AHU 3 according to the present embodiment will be described.

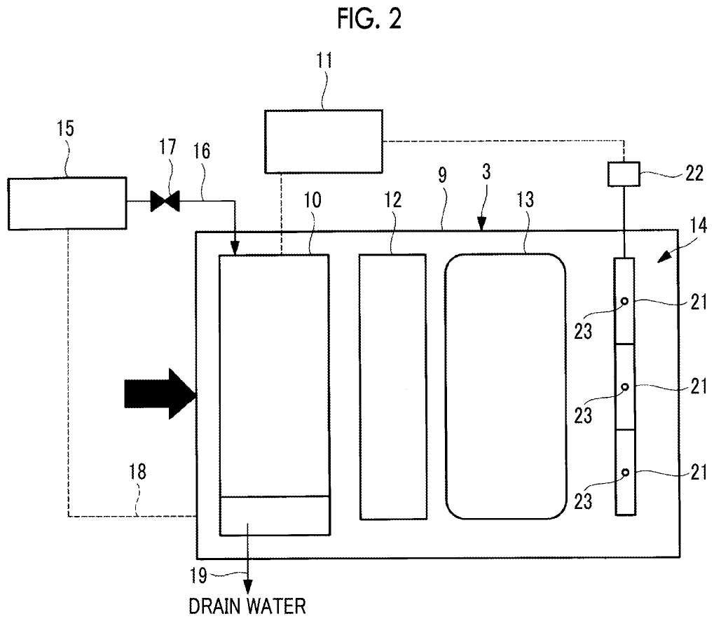

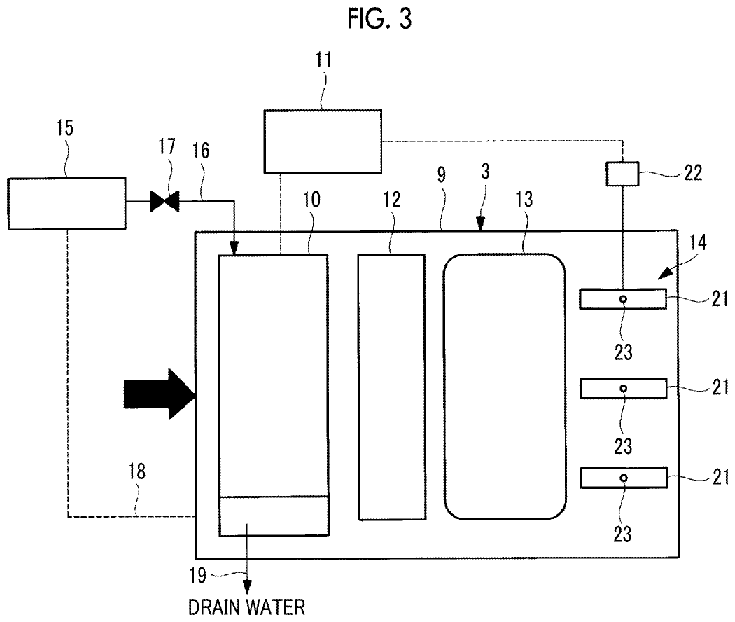

As illustrated in FIGS. 2 and 3, the AHU 3 has, for example, the electrical precipitator unit 10, a control unit 11, the filter unit 12, the air conditioning unit 13, an ozone removal unit 14, and so on. The electrical precipitator unit 10, the filter unit 12, and the air conditioning unit 13 are installed inside the casing 9 of the AHU 3, and the air taken into the AHU 3 flows in the order of the electrical precipitator unit 10, the filter unit 12, the air conditioning unit 13, and the ozone removal unit 14. The treatment speed in the AHU 3 is, for example, in the range of 2.5 m/s to 3.5 m/s applied in a normal AHU. In the AHU 3, the electrical precipitator unit 10, the filter unit 12, and the ozone removal unit 14 configure the air purification device according to the present disclosure.

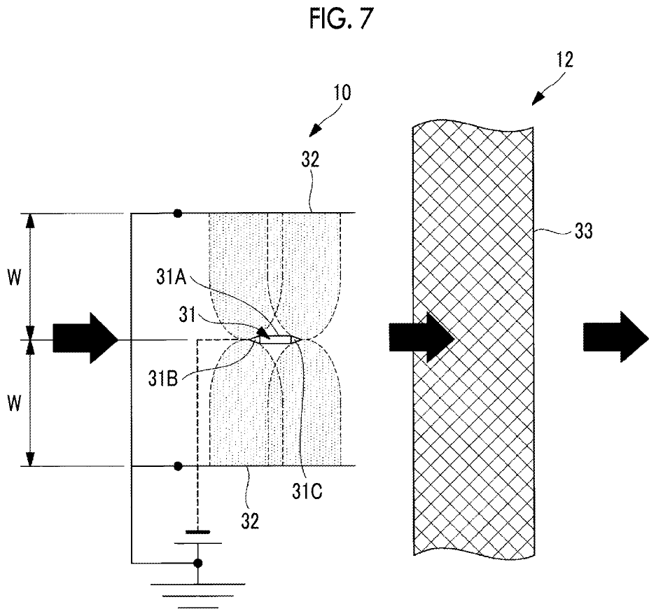

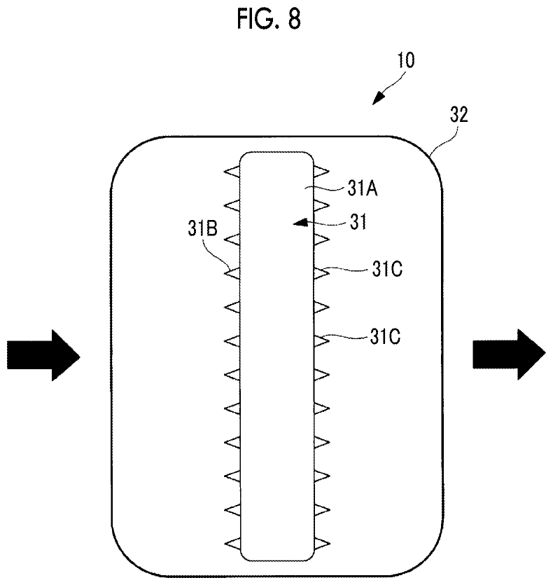

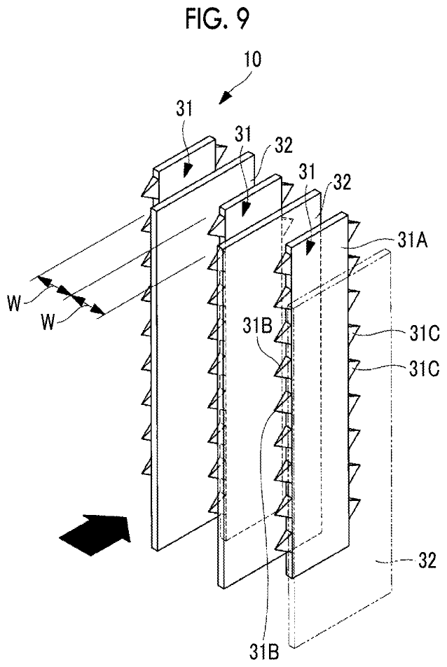

The electrical precipitator unit 10 removes dust (including particulate matter) contained in the air taken in by the air conditioning device 1. As illustrated in FIGS. 7 to 9, the electrical precipitator unit 10 includes discharge electrodes 31 charging particles, collecting electrodes 32 installed so as to face the discharge electrodes 31, and so on. By corona discharge occurring at the discharge electrode 31, gas molecules are ionized, and the particles contained in the air are energized as the particles pass through the electric field between the electrodes. Then, the charged particles are attached at the collecting electrode 32 and collected.

The control unit 11 controls the ozone removal unit 14 to switch between a first mode in which the ozone removal unit 14 removes ozone and a second mode in which the ozone removal unit 14 stops removing ozone. The control unit 11 transmits a control signal for controlling, for example, the start or stop of the operation of the ozone removal unit 14 to the ozone removal unit 14.

The control unit 11 adjusts the voltage or energization condition applied to the electrical precipitator unit 10. The control unit 11 transmits a control signal for voltage or energization condition adjustment to the electrical precipitator unit 10.

The control unit 11 is configured by, for example, a central processing unit (CPU), a random access memory (RAM), a read only memory (ROM), a computer-readable storage medium, and so on. A series of processing for realizing various functions is, for example, stored in the storage medium or the like in the form of a program, the CPU reads this program into the RAM or the like to execute information and/or arithmetic processing, and the various functions are realized as a result. The program may be applied in the form of being, for example, pre-installed in the ROM or another storage medium, provided in a state of being stored in the computer-readable storage medium, or distributed via wired or wireless communication means. The computer-readable storage medium is a magnetic disk, a magneto-optical disk, a CD-ROM, a DVD-ROM, a semiconductor memory, or the like.

The filter unit 12 is installed on the downstream side of the electrical precipitator unit 10 and removes dust contained in the air that has passed through the electrical precipitator unit 10. A medium-efficiency particulate air filter 33 normally used in the AHU 3 can be applied to the filter unit 12. The medium-efficiency particulate air filter 33 is, for example, a sheet member and has a pleated multi-fold structure. In the present embodiment, the filter unit 12 is optional. A coarse filter may be installed instead of the medium-efficiency particulate air filter 33. The presence or absence of the filter unit 12 and the type of the filter are appropriately selected in accordance with particulate matter removal performance. The medium-efficiency particulate air filter 33 may be a filter with a pre-charged filter material or may be a filter with a filter material that is not pre-charged.

The medium-efficiency particulate air filter applied as the medium-efficiency particulate air filter 33 in the present embodiment is defined in JIS terminology as having medium degree particle collection efficiency mainly with respect to small particles of 5 μm or less. According to general literature as to the performance of a medium-efficiency particulate air filter, the efficiency of collection of particles with a medium diameter of 1.6 μm to 2.3 μm is approximately 50% to 80% by the so-called colorimetric method and the efficiency of collection by the DOP method (0.3 μm particle) is approximately 15% to 50%. The inventor has found regarding the medium-efficiency particulate air filter that in an experiment using atmospheric dust that does not show characteristic of attachment as in the case of DOP particles, even 0.4 μm particles show a collectability of as low as approximately 15% to 25% and most fine submicron particles slip through.

The air conditioning unit 13 performs temperature and/or humidity adjustment of the air that has passed through the electrical precipitator unit 10 and the filter unit 12 and supplies the adjusted air to the space 50. The air conditioning unit 13 has a heat exchanger, a humidifier, and so on.

A case where the AHU 3 described above is an integrated AHU in which the electrical precipitator unit 10 is installed inside the casing 9 has been described and yet the present disclosure is not limited to this example. In an example of the AHU 3, the filter unit 12 and the air conditioning unit 13 may be installed inside a casing and the electrical precipitator unit 10 and the ozone removal unit 14 may be attached to the outside of the casing (not illustrated). In other words, the air handling unit includes one in which the electrical precipitator unit 10 and the ozone removal unit 14 are externally attached with respect to a configuration packaged without incorporating the electrical precipitator unit 10 and the ozone removal unit 14. Accordingly, the air conditioning device according to the present disclosure can be applied to both the newly installed AHU 3 incorporating the electrical precipitator unit 10 and the AHU 3 at which the electrical precipitator unit 10 and the ozone removal unit 14 are additionally installed.

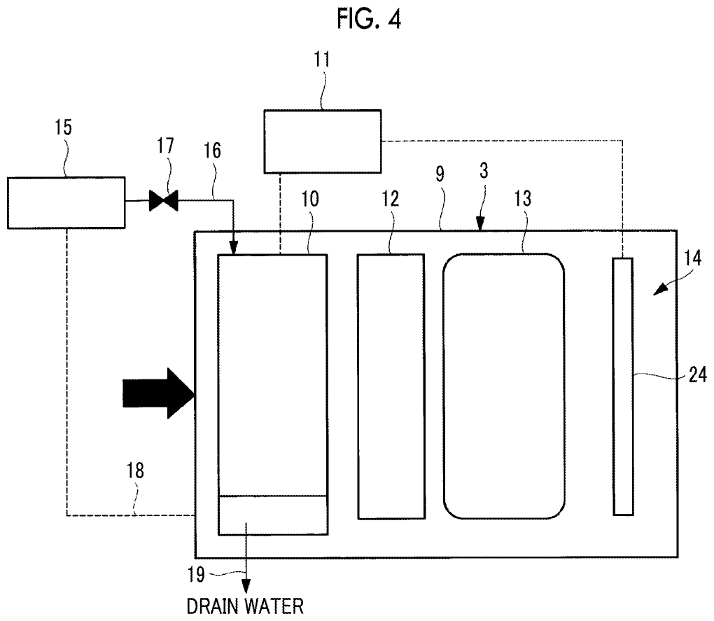

The ozone removal unit 14 is installed on the downstream side of the filter unit 12 and is capable of removing ozone contained in flowing air. The ozone removal unit 14 may be, for example, a removal main body portion 21 having a filter carrying an ozone decomposition catalyst as illustrated in FIGS. 2 and 3 or may be an ultraviolet lamp 24 emitting ultraviolet rays capable of decomposing ozone as illustrated in FIG. 4.

The ozone removal unit 14 is controlled by the control unit 11 to switch between the first mode and the second mode. In the first mode, ozone is removed by the ozone removal unit 14 and no ozone is supplied to the space supplied with the air that has passed through the electrical precipitator unit 10 and the filter unit 12. In the second mode, ozone removal by the ozone removal unit 14 is stopped and ozone generated by corona discharge at the discharge electrode 31 is supplied to the space supplied with the air that has passed through the electrical precipitator unit 10 and the filter unit 12.

Next, the ozone removal unit 14 according to the present embodiment will be described.

<Ozone Removal Unit (First Example)>

The ozone removal unit 14 according to a first example has a filter carrying an ozone decomposition catalyst. As illustrated in FIGS. 2 and 3, the ozone removal unit 14 has the filter-shaped removal main body portion 21 carrying an ozone decomposition catalyst and a drive unit 22 driving the removal main body portion 21.

The removal main body portion 21 is, for example, a filter with a plurality of honeycomb-shaped openings that carries an ozone decomposition catalyst. Examples of the ozone decomposition catalyst include manganese dioxide and nickel oxide. As a result, although ozone is generated by corona discharge at the discharge electrode 31, the ozone removal unit 14 installed downstream of the filter unit 12 is capable of removing ozone contained in the air that passes through the removal main body portion 21.

The drive unit 22 is controlled by the control unit 11 to drive the removal main body portion 21 and change the position or direction of the removal main body portion 21. In the first mode, the removal main body portion 21 is in a position or direction in which the air that has passed through the electrical precipitator unit 10 and the filter unit 12 passes through the removal main body portion 21. As a result, ozone contained in the air passing through the removal main body portion 21 is removed.

In the second mode, the removal main body portion 21 is in a position or direction in which the air that has passed through the electrical precipitator unit 10 and the filter unit 12 does not pass through the removal main body portion 21. As a result, no air passes through the removal main body portion 21, and thus the ozone-containing air that has passed through the electrical precipitator unit 10 and the filter unit 12 is supplied to the downstream side. For example, ozone generated by corona discharge at the discharge electrode 31 is supplied to the space supplied with the air that has passed through the electrical precipitator unit 10 and the filter unit 12.

Next, the configuration and operation of the ozone removal unit 14 according to the first example will be described.

The removal main body portion 21 has a frame rectangular in a front view. As illustrated in FIG. 2, in the first mode, the surface of the removal main body portion 21 is installed so as to intersect the flow path in the casing 9 of the AHU 3. As illustrated in FIG. 3, in the second mode, the surface of the removal main body portion 21 is installed so as to be parallel to the flow path.

A rotary shaft 23 or the like is installed in the removal main body portion 21, and the removal main body portion 21 is rotatably supported about the rotary shaft 23 installed in the removal main body portion 21. The drive unit 22 drives the removal main body portion 21 to rotate the removal main body portion 21.

As a result, the removal main body portion 21 is rotated by the drive unit 22 about the rotary shaft 23 installed in the removal main body portion 21 in a supported state. By changing the direction of the removal main body portion 21 to intersect the flow path in the first mode, the removal main body portion 21 has a direction in which the air that has passed through the electrical precipitator unit 10 and the filter unit 12 passes through the removal main body portion 21. By changing the direction of the removal main body portion 21 to be parallel to the flow path in the second mode, the removal main body portion 21 has a direction in which the air that has passed through the electrical precipitator unit 10 and the filter unit 12 does not pass through the removal main body portion 21.

<Ozone Removal Unit (Modification Example of First Example)>

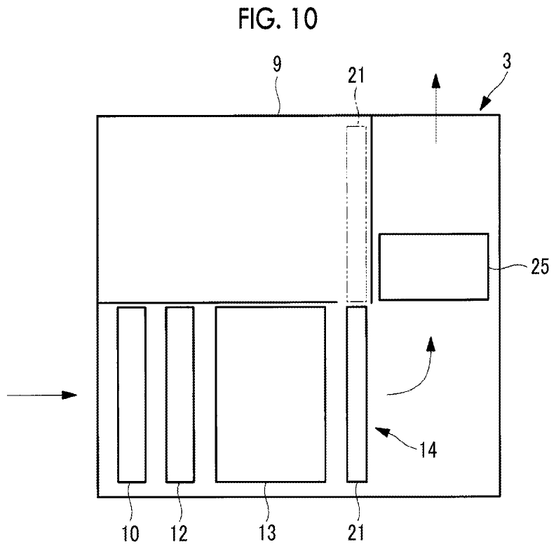

The removal main body portion 21 of the ozone removal unit 14 is not limited to the case of having a configuration rotatable in the flow path. For example, the removal main body portion 21 may have a slidable configuration as illustrated in FIG. 10. As for the AHU 3 illustrated in FIG. 10, an example in which a blower 25 is installed on the downstream side of the ozone removal unit 14 is illustrated.

The removal main body portion 21 has a frame rectangular in a front view. In the first mode, the surface of the removal main body portion 21 is installed so as to be positioned to intersect the flow path in the casing of the AHU 3 (removal main body portion 21 indicated by solid line in FIG. 10). In the second mode, the surface of the removal main body portion 21 is installed so as to be positioned away from the flow path (removal main body portion 21 indicated by two-dot chain line in FIG. 10).

The removal main body portion 21 is supported in both end portions by, for example, rails so as to be movable in the direction parallel to the plane direction of the removal main body portion 21. The drive unit drives the removal main body portion 21 to move the removal main body portion 21.

As a result, the removal main body portion 21 is moved by the drive unit in the direction parallel to the plane direction of the removal main body portion 21. By the removal main body portion 21 being moved to and installed at a position intersecting the flow path in the first mode, the removal main body portion 21 has a position where the air that has passed through the electrical precipitator unit 10 and the filter unit 12 passes through the removal main body portion 21. Accordingly, ozone contained in the air passing through the removal main body portion 21 is removed.

By the removal main body portion 21 being moved to and installed at a position away from the position intersecting the flow path in the second mode, the removal main body portion 21 has a position where the air that has passed through the electrical precipitator unit 10 and the filter unit 12 does not pass through the removal main body portion 21. Accordingly, since the air does not pass through the removal main body portion 21, the ozone-containing air that has passed through the electrical precipitator unit 10 and the filter unit 12 is supplied to the downstream side.

As for the removal main body portion 21, only one frame (filter) may be installed in the flow path or a plurality of frames (filters) may be installed in a certain flow path cross section. FIGS. 2 and 3 illustrate an example in which the removal main body portion 21 that has a plurality of frames is installed and each frame has the rotary shaft 23 and is rotatable.

<Ozone Removal Unit (Second Example)>

As illustrated in FIG. 4, the ozone removal unit 14 according to a second example is the ultraviolet lamp 24 that emits ultraviolet rays. The ultraviolet rays emitted by the ultraviolet lamp 24 decompose ozone, and ozone is removed from the air that has passed through the vicinity of the ozone removal unit 14.

The ultraviolet lamp 24 is controlled by the control unit 11 to switch between the first mode and the second mode. In the first mode, the ultraviolet lamp 24 irradiates the air that has passed through the electrical precipitator unit 10 and the filter unit 12 with ultraviolet rays. As a result, ozone contained in the air passing through the flow path is removed.

In the second mode, the ultraviolet lamp 24 stops irradiating the air that has passed through the electrical precipitator unit 10 and the filter unit 12 with ultraviolet rays. As a result, no ozone is decomposed by ultraviolet rays, and thus the ozone-containing air that has passed through the electrical precipitator unit 10 and the filter unit 12 is supplied to the downstream side. For example, ozone generated by corona discharge at the discharge electrode 31 is supplied to the space supplied with the air that has passed through the electrical precipitator unit 10 and the filter unit 12.

<Ozone Removal Unit (Third Example)>

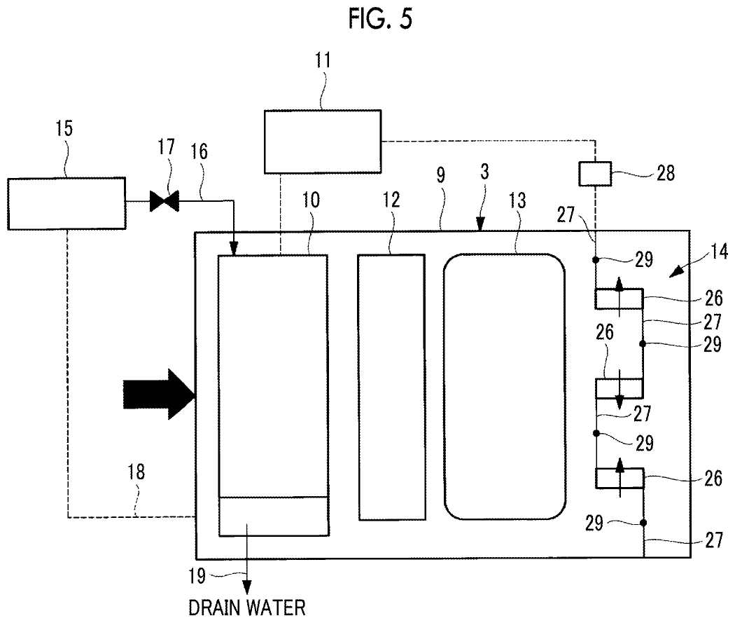

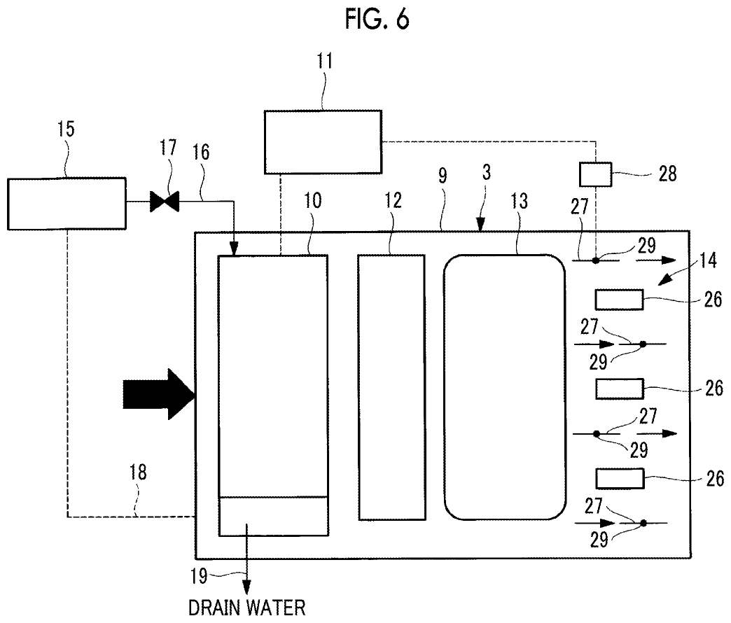

The ozone removal unit 14 according to a third example has a filter carrying an ozone decomposition catalyst. As illustrated in FIGS. 5 and 6, the ozone removal unit 14 has a filter-shaped removal main body portion 26 carrying an ozone decomposition catalyst, a damper 27 changing the flow direction of air, and a drive unit 28 driving the damper 27.

The removal main body portion 26 is, for example, a filter with a plurality of honeycomb-shaped openings that carries an ozone decomposition catalyst. Examples of the ozone decomposition catalyst include manganese dioxide and nickel oxide. As a result, although ozone is generated by corona discharge at the discharge electrode 31, the ozone removal unit 14 installed downstream of the filter unit 12 is capable of removing ozone contained in the air that passes through the removal main body portion 26.

The removal main body portion 26 has a frame rectangular in a front view, and the surface of the removal main body portion 26 is installed so as to be parallel to the flow path. One or more removal main body portions 26 are installed. In a case where two or more removal main body portions 26 are installed, the removal main body portions 26 are installed in a direction intersecting the flow path.

The damper 27 is installed between the removal main body portion 26 and the inner wall of the casing 9 or between two adjacent removal main body portions 26. The damper 27 is a plate-shaped member rectangular in a front view and can be rotated by the drive unit 28. The damper 27 is installed with, for example, a rotary shaft 29, and the damper 27 is supported so as to be rotatable about the rotary shaft 29 installed on the damper 27.

As illustrated in FIG. 5, in the first mode, the damper 27 is installed such that the surface of the damper 27 intersects the flow path in the casing 9 of the AHU 3. The damper 27 installed on one surface side of the removal main body portion 26 blocks the flow path on the upstream side of the removal main body portion 26, and the damper 27 installed on the other surface side of the removal main body portion 26 blocks the flow path on the downstream side of the removal main body portion 26. As a result, it is ensured that the air flowing in the casing 9 flows along the flow path formed by the damper 27 and passes through the removal main body portion 26.

As illustrated in FIG. 6, in the second mode, the damper 27 is installed such that the surface of the damper 27 is parallel to the flow path. As a result, unlike in the first mode, the air flowing in the casing 9 flows along the surface of the removal main body portion 26 parallel to the flow path without being interrupted by the damper 27 and is unlikely to pass through the removal main body portion 26.

The drive unit 28 is controlled by the control unit 11 to drive the damper 27 and change the direction of the damper 27. In other words, the drive unit 28 drives the damper 27 to rotate the damper 27. In the first mode, the damper 27 is directed to pass the air that has passed through the electrical precipitator unit 10 and the filter unit 12 through the removal main body portion 26. As a result, ozone contained in the air passing through the removal main body portion 26 is removed.

In the second mode, the damper 27 is directed not to pass the air that has passed through the electrical precipitator unit 10 and the filter unit 12 through the removal main body portion 26. As a result, no air passes through the removal main body portion 26, and thus the ozone-containing air that has passed through the electrical precipitator unit 10 and the filter unit 12 is supplied to the downstream side. For example, ozone generated by corona discharge at the discharge electrode 31 is supplied to the space supplied with the air that has passed through the electrical precipitator unit 10 and the filter unit 12.

The damper 27 is rotated by the drive unit 28 about the rotary shaft 29 installed on the damper 27. By changing the direction of the damper 27 to intersect the flow path in the first mode, the removal main body portion 26 has a direction in which the air that has passed through the electrical precipitator unit 10 and the filter unit 12 passes through the removal main body portion 26. By changing the direction of the removal main body portion 26 to be parallel to the flow path in the second mode, the removal main body portion 26 has a direction in which the air that has passed through the electrical precipitator unit 10 and the filter unit 12 does not pass through the removal main body portion 26.

<Ozone Removal Unit (Modification Example of Third Example)>

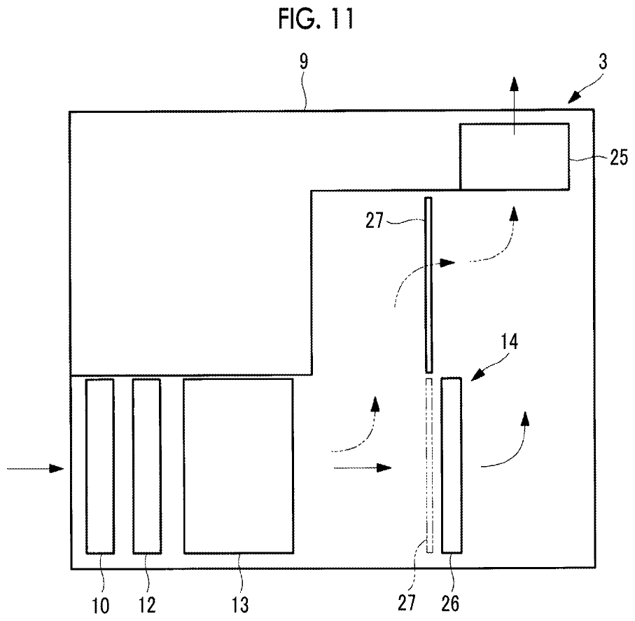

The damper 27 of the ozone removal unit 14 is not limited to the case of having a configuration rotatable in the flow path. For example, the damper 27 may have a slidable configuration as illustrated in FIG. 11. As for the AHU 3 illustrated in FIG. 11, an example in which the blower 25 is installed on the downstream side of the ozone removal unit 14 is illustrated.

The damper 27 is a plate-shaped member rectangular in a front view. In the first mode, the surface of the damper 27 does not block the surface of the removal main body portion 26 and is installed so as to be positioned away from the removal main body portion 26 (damper 27 indicated by solid line in FIG. 11). In the second mode, the surface of the damper 27 blocks the surface of the removal main body portion 26 and is installed so as to be at a position where a flow path that does not pass through the removal main body portion 26 is formed (damper 27 indicated by two-dot chain line in FIG. 11).

The damper 27 is supported in both end portions by, for example, rails so as to be movable in the direction parallel to the plane direction of the damper 27. The drive unit drives the damper 27 to move the damper 27.

As a result, the damper 27 is moved by the drive unit in the direction parallel to the plane direction of the damper 27. By the damper 27 being moved to and installed at a position away from the removal main body portion 26 without the surface of the damper 27 blocking the surface of the removal main body portion 26 in the first mode, the damper 27 has a position where the air that has passed through the electrical precipitator unit 10 and the filter unit 12 passes through the removal main body portion 26. As a result, ozone contained in the air passing through the removal main body portion 26 is removed.

In the second mode, the damper 27 is moved to and installed at a position where the surface of the damper 27 blocks the surface of the removal main body portion 26 and a flow path that does not pass through the removal main body portion 26 is formed. As a result, the damper 27 has a position where the air that has passed through the electrical precipitator unit 10 and the filter unit 12 does not pass through the removal main body portion 26. As a result, no air passes through the removal main body portion 26, and thus the ozone-containing air that has passed through the electrical precipitator unit 10 and the filter unit 12 is supplied to the downstream side.

<Electrical Precipitator Unit>

Next, the electrical precipitator unit 10 of the AHU 3 according to the present embodiment will be described with reference to FIGS. 7 to 9.

In the electrical precipitator unit 10, gas flows in one direction from the upstream side to the downstream side of the AHU 3.

The collecting electrodes 32, which are, for example, metallic plate-shaped members, are installed in the electrical precipitator unit 10. The collecting electrode 32 has a plate surface provided parallel to the gas flow direction. The plurality of collecting electrodes 32 are installed at predetermined intervals in a direction orthogonal to the gas flow direction. The collecting electrode 32 is, for example, an opening portion-less flat plate-shaped member, a reticulated member having an opening portion, a punching metal, or the like.

The discharge electrode 31 is installed between the collecting electrodes 32 that are adjacent to each other. The discharge electrode 31 has a main body portion 31A and corona discharge portions 31B and 31C, and the corona discharge portions 31B and 31C are provided so as to protrude from the main body portion 31A. The corona discharge portions 31B and 31C have, for example, a spiny shape.

At least one discharge electrode 31 may be provided, and the corona discharge portions are two or more stages in total in the gas flow direction. In the example illustrated in FIGS. 7 to 9, one discharge electrode 31 is installed. The main body portion 31A of the discharge electrode 31 is a long plate-shaped member that is long in one direction. The plate surface of the main body portion 31A may be provided with, for example, circular openings (through-holes) at predetermined intervals along the length direction and the main body portion 31A may be an opening-less flat plate. Two or more discharge electrodes 31 may be provided and, in this case, the corona discharge portions are four or more stages in total.

The main body portion 31A has a plate surface provided parallel to the gas flow direction. The main body portion 31A is installed such that the length direction of the main body portion 31A is orthogonal to the gas flow direction and orthogonal to the direction in which the plurality of collecting electrodes 32 are installed.

The corona discharge portion 31B protrudes toward the upstream side in the gas flow direction in one side end portion of the main body portion 31A, for example, the upstream side end portion in the gas flow direction. The corona discharge portion 31B is an example of a first corona discharge portion. The corona discharge portion 31C protrudes toward the downstream side in the gas flow direction in the other side end portion of the main body portion 31A, for example, the downstream side end portion in the gas flow direction. The corona discharge portion 31C is an example of a second corona discharge portion.

Corona discharge occurs at the corona discharge portions 31B and 31C, and ionic wind is generated from the tips of the corona discharge portions 31B and 31C toward the facing collecting electrode 32 side. In other words, the discharge electrode 31 can be subjected to corona discharge from the corona discharge portions 31B and 31C toward the collecting electrode 32 to allow ionic wind to flow.

In the electrical precipitator unit 10, a total of two stages of corona discharge portions are provided in the gas flow direction with the corona discharge portion 31B provided on the upstream side and the corona discharge portion 31C provided on the downstream side in the discharge electrode 31.

An interval W between the surface of the discharge electrode 31 and the surface of the collecting electrode 32 is set in the range of, for example, 10 mm or more and 40 mm or less. In a general electrical precipitator, discharge and collecting electrodes have an interval in the range of 150 mm or more and 250 mm or less. In other words, the interval W between the discharge electrode 31 and the collecting electrode 32 is relatively narrow. In a case where the interval W between the discharge electrode 31 and the collecting electrode 32 is narrow, the collecting area per unit amount can be increased. However, at the interval W that is too small, the dust collected by the collecting electrode 32 may cause local electric field concentration. Accordingly, the interval W that is ensured is preferably 10 mm or more.

In the present embodiment, collection performance improvement is achieved since the corona discharge portions 31B and 31C are provided in a plurality of stages. In existing air purification devices, corona current is suppressed as much as possible for ozone generation suppression. In addition, of the existing air purification devices, an electrical precipitator is configured based on dust charging in a charging portion and collection by Coulomb force under the electric field on the downstream side of the charging portion regarding dust collection. Of the existing air purification devices, an electrostatic filter is configured based on dust charging in a charging portion and collection by Coulomb force acting by the charge of the particles in a filter on the downstream side of the charging portion regarding dust collection. Accordingly, in each case, the charging portion is provided at only one place from the viewpoint of suppressing ozone generation and from the viewpoint that collection by Coulomb force on the downstream side of the charging portion being possible is enough.

On the other hand, in the electrical precipitator unit 10 according to the present embodiment, corona discharge is performed more stably than in the case of a positive energization by negative energization application to the discharge electrode 31. The electrical precipitator unit 10 is configured based on dust charging and collection by corona current continuation regarding dust collection, and collection is performed inside the electrical precipitator unit 10 as well. In the electrical precipitator unit 10, ionic wind is sustained by dust being charged and corona current being ensured, and the ionic wind also promotes dust collection. Collection using ionic wind is also realized since the corona discharge portions 31B and 31C are provided in a plurality of stages along the gas flow direction (two stages in the example illustrated in FIGS. 7 to 9).

The electrical precipitator unit 10 is configured to actively generate ozone. With the generated ozone, it is possible to deodorize the air in the space 50, inactivate a virus contained in the air, and sterilize fungi. As for existing air purification devices, ozone generation suppression is a task. On the other hand, in the present embodiment, the amount of ozone generation is adjusted by adjusting the voltage or energization condition applied to the discharge electrode 31 of the electrical precipitator unit 10 based on ozone concentration and environmental conditions.

The medium-efficiency particulate air filter 33 has a low pressure loss and a large dust holding capacity. The electrical precipitator unit 10 is installed upstream of the filter unit 12, dust is collected at the electrical precipitator unit 10 as well, and thus the amount of dust collected by the medium-efficiency particulate air filter 33 and the frequency of replacement of the medium-efficiency particulate air filter 33 are reduced. At the electrical precipitator unit 10 on the upstream side, a sufficient amount of charge can be applied to particles in a diffusion charging process (for example, submicron particles) by the plurality of stages of corona discharge portions 31B and 31C (two stages along the gas flow direction in the example illustrated in FIGS. 7 to 9), and thus a strong electrostatic force acts on the main body of the medium-efficiency particulate air filter 33. As a result, the collection efficiency of the filter unit 12, fine particle collection efficiency in particular, is significantly improved. The medium-efficiency particulate air filter 33 may be a filter with a pre-charged filter material and, in this case, the collection efficiency can be further improved.

The discharge electrode 31 is connected to a power supply having a negative polarity, and the collecting electrode 32 is grounded and has a positive polarity. Stable discharge is possible in a case where a negative energization is applied to the discharge electrode 31. It is easy to generate ozone at the time of discharge by applying a negative energization to the discharge electrode 31. The present disclosure is not limited to this example, a positive energization may be applied to the discharge electrode 31, and the collecting electrode 32 may be used as a negative electrode.

<Control of Ozone Removal Unit>

Next, the control of the ozone removal unit 14 according to the present embodiment will be described.

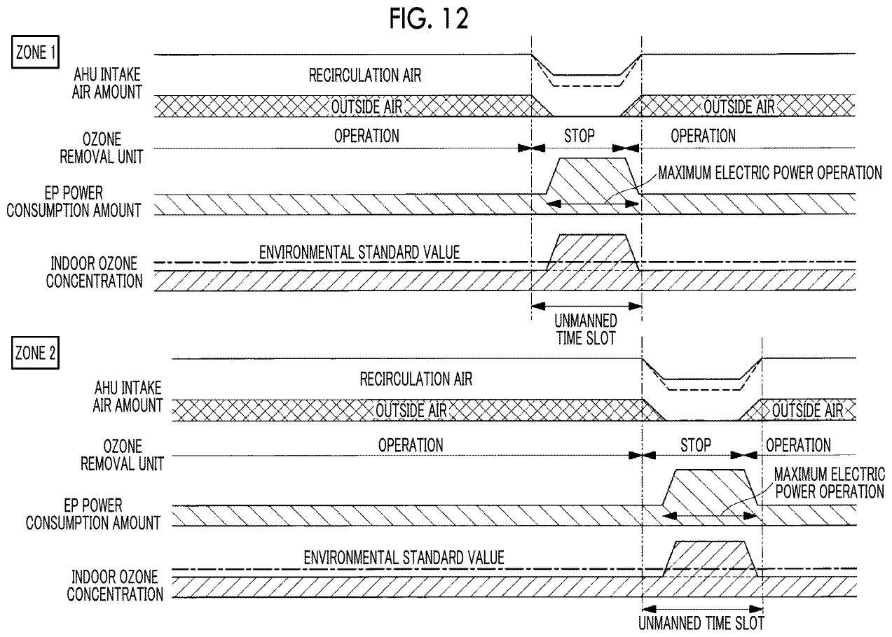

For example, as illustrated in FIG. 12, in a time slot when a person stays in the space 50, a switch to the first mode is performed with the air conditioning device 1 in operation and ozone removal by the ozone removal unit 14 is started such that the ozone concentration becomes equal to or less than the environmental standard value. As a result, the ozone concentration is reduced to the extent that ozone does not adversely affect the person staying in the space. On the other hand, when no one stays in the space 50 or in a state where no one is allowed to enter the space 50, a switch to the second mode is performed and ozone removal by the ozone removal unit 14 is stopped such that the ozone concentration becomes a high value. As a result, the ozone concentration is increased such that the space supplied with air is forcibly deodorized or sterilized by ozone.

When a switch to the second mode is performed in a time slot when no one stays in the space 50 or in a state where no one is allowed to enter the space 50, the outside air intake at the AHU 3 may be stopped and the ratio of the amount of recirculation air to the total intake air amount of the AHU 3 may be caused to reach 100% (see the AHU intake amount in FIG. 12) so that the ozone concentration is efficiently increased. When the outside air intake at the AHU 3 is stopped after a switch to the second mode, the amount of recirculation air may be reduced more than the amount of recirculation air in the first mode (see the broken line portion of the AHU intake amount in FIG. 12). As a result, the ozone concentration of the air supplied from the AHU 3 to the space 50 can be increased.

Further, a predetermined CT value may be ensurable so that the effect of sterilization or deodorization in the space 50 is obtained. The CT value is a value (ppm·min) expressed by the product of the ozone concentration (ppm) and the time (min) of contact with an object to be treated at the ozone concentration. Accordingly, even in a case where the ozone concentration is low, by setting a long contact time, it is possible to ensure a CT value equivalent to that in the case of contact with a high ozone concentration in a short time. For example, the second mode may be set to a relatively long time in a case where the absolute ozone concentration is suppressed or in a case where there is a limit to the amount of ozone generated at the electrical precipitator unit 10.

For example, an ozone concentration measuring unit is installed in the space 50 supplied with temperature-adjusted air, that is, the air that has passed through the electrical precipitator unit 10 and the filter unit 12 from the AHU 3. The ozone concentration measuring unit measures the ozone concentration in the space. Data related to the measured ozone concentration is transmitted from the ozone concentration measuring unit to the control unit 11. The control unit 11 receives a signal related to the measurement data from the ozone concentration measuring unit. After a switch to the second mode, the control unit 11 determines whether or not a predetermined CT value has been exceeded based on the measured ozone concentration and the time of measurement of the ozone concentration. The control unit 11 switches from the second mode to the first mode when it is determined that the predetermined CT value has been exceeded and continues the second mode when it is determined that the predetermined CT value is not exceeded.

In an office building or a large space where people cannot enter depending on the time slot (for example, a theater), it is also possible to automatically switch between the first mode and the second mode using an unmanned time slot at night.

In the case of returning from a state where the ozone concentration in the space 50 is high to a state where the ozone concentration is low and satisfies the environmental standard value, a switch is performed from the second mode of high concentration to the first mode of low concentration. At this time, the return can be expedited by temporarily increasing the amount of outside air taken into the AHU 3 as compared with the normal operation. At this time, the operation of the electrical precipitator unit 10 may be stopped in order to efficiently reduce the ozone concentration. As a result, the ozone removal unit 14 acts only to remove the ozone in the space. In a case where the ozone removal unit 14 is of high removal capacity, the operation of the electrical precipitator unit 10 does not necessarily have to be stopped.

In a case where the plurality of AHUs 3 are installed and the AHUs 3 supply air to different spaces 50, as illustrated in FIG. 12, a switch to the second mode is performed for each space 50 and a target space is deodorized or sterilized. For example, in a case where deodorization or sterilization is performed in one space 50 (Zone 1 in the example illustrated in FIG. 12), only the target space is set to the second mode and the other space 50 (Zone 2 in the example illustrated in FIG. 12) remains in the first mode.

In the space 50 set to the second mode, the flow of outside air into the space 50 is blocked, the amount of recirculation air is set to 100%, and ozone removal by the ozone removal unit 14 is stopped. As a result, the target space is deodorized or sterilized.

At this time, the other space 50 may remain in normal operation in a case where the recirculation air line that connects the AHU 3 and the space 50 is independent for each space 50. On the other hand, in a case where the recirculation air line is common and the air in the plurality of spaces 50 is suctioned in and returned to the AHU 3, the amount of recirculation air in the target space is increased and the amount of recirculation air in the other space 50 is decreased. As a result, the amount of recirculation air in the target space supplied from the AHU 3 increases, and thus the ozone concentration can be efficiently increased.

<Control of Electrical Precipitator Unit>

Next, the control of the electrical precipitator unit 10 according to the present embodiment will be described.

The control unit 11 adjusts the voltage or energization condition applied to the discharge electrode 31 of the electrical precipitator unit 10. As a result, the amount of ozone generated as a result of the corona discharge at the discharge electrode 31 is adjusted. In a case where the ozone concentration in the space 50 is increased, the control unit 11 changes the voltage or energization condition applied to the discharge electrode 31 of the electrical precipitator unit 10 to increase the input power. On the other hand, in a case where the ozone concentration in the space 50 is lowered, the control unit 11 changes the voltage or energization condition applied to the discharge electrode 31 of the electrical precipitator unit 10 to reduce the input power. The energization is paused as needed.

The control unit 11 applies a continuous energization condition or an intermittent energization condition in a case where the control unit 11 adjusts the energization condition applied to the discharge electrode 31 of the electrical precipitator unit 10.

As for the continuous energization condition, full-wave rectification is performed in a direct-current high-voltage power supply device (transformer rectifier), and direct current is applied to the discharge electrode 31. Voltage level adjustment is performed by an increase or decrease in supply current on the primary side. The current attributable to corona discharge and flowing through the electrical precipitator unit 10 also increases or decreases in accordance with the voltage level, the amount of ozone generation changes, and the ozone concentration changes. It is possible to switch between energization ON and OFF in the case of the continuous energization condition as well as the intermittent energization to be described later. The ON-OFF switching of the continuous energization condition is controlled by, for example, an external timer and switching time is on the order of at least a few seconds.

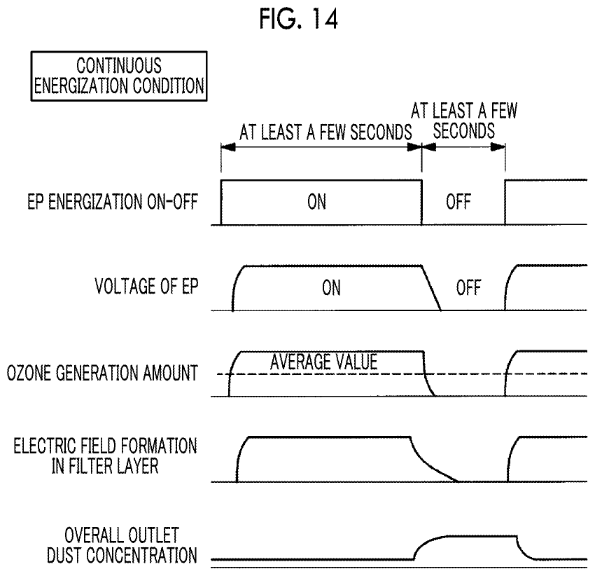

In the continuous energization condition, as illustrated in FIG. 14, the amount of ozone generation increases as the voltage and current increase by the energization of the electrical precipitator unit 10 being turned ON and the amount of ozone generation decreases as the voltage and current decrease by the energization of the electrical precipitator unit 10 being turned OFF. With the energization of the electrical precipitator unit 10 OFF, the dust passing through the electrical precipitator unit 10 is not charged, and thus no charged dust flies to the filter unit 12 on the downstream side. As a result, at the time of energization OFF, no electric field is formed in the medium-efficiency particulate air filter 33, no charge can be maintained, and thus the dust concentration at the outlet on the downstream side of the filter unit 12 tends to increase.

On the other hand, as for the intermittent energization condition, in a commercial frequency-based direct-current high-voltage power supply device (transformer rectifier), the output on the primary side of the transformer is intermittently turned OFF. For example, the energizing rate is reduced to 1/3 by turning ON (adopting) one out of three mountains and turning OFF the other two. As for energization at this time, the energization is turned ON and OFF in units of 10 milliseconds in an area of, for example, 50 Hz, and thus the ON timing is every 30 milliseconds and ON and OFF are repeated in a case where the energizing rate is 1/3. In the case of a high-frequency power supply or energization condition by a boosting voltage method using an electronic circuit, control in finer frequency units is possible and, in that case, control for turning ON the energization every 1 to 3 milliseconds and repeating ON and OFF is also possible. Then, the amount of ozone generation changes in accordance with the energizing rate, and the ozone concentration changes.

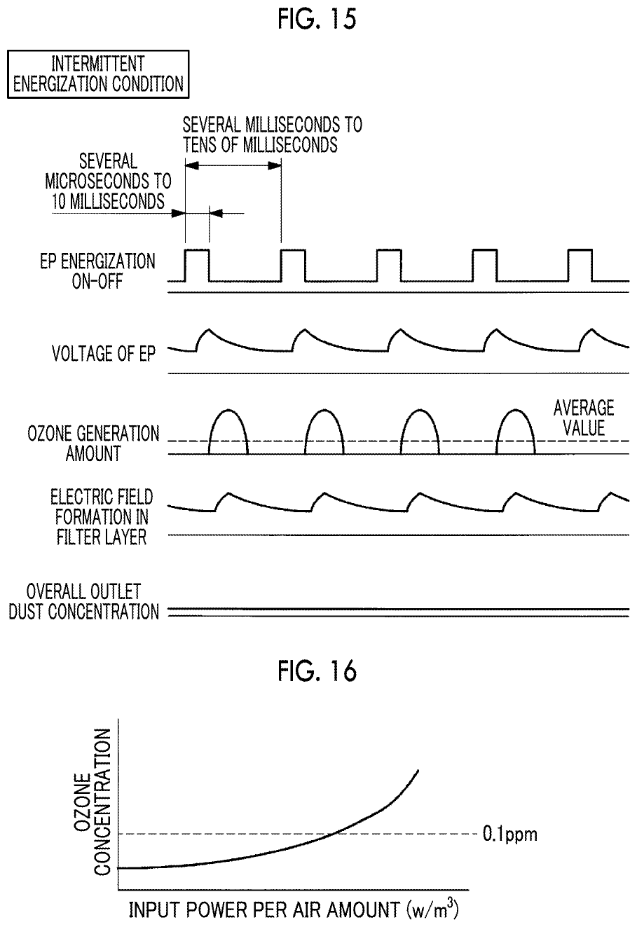

In the intermittent energization condition, as illustrated in FIG. 15, with the energization of the electrical precipitator unit 10 ON, charging current flows through the capacitor component of the electrical precipitator unit 10 to lead to a rise in voltage and current flows as a result of corona discharge, and then discharge current flows and the voltage gradually decreases in the duration of an OFF state where no new energization is turned ON. Also in the intermittent energization condition, the amount of ozone generation increases as the voltage and current increase by the energization of the electrical precipitator unit 10 being turned ON and the amount of ozone generation decreases as the voltage and current decrease by the energization of the electrical precipitator unit 10 being turned OFF. Even with the energization of the electrical precipitator unit 10 OFF once, the cycle is short in the intermittent energization condition, and thus the energization is turned ON again during dust passage through the electrical precipitator unit 10 as well and, as a result, energization is turned ON and OFF many times and the dust itself is always charged. Accordingly, charged dust always flies to the filter unit 12 on the downstream side. As a result, a certain amount of charge is held in the medium-efficiency particulate air filter 33, and thus a situation is maintained in which the dust concentration at the outlet on the downstream side of the filter unit 12 is stably reduced.

By the intermittent energization condition as compared with the continuous energization condition, electric power can be reduced, energy can be saved, and high filter performance can be maintained by the electric field in the filter unit 12 being maintained. The input power can be suppressed to a low level, and thus the ozone concentration can also be suppressed to a low level.

The adjustment of the ozone concentration in the space 50 by the electrical precipitator unit 10 is performed by switching in accordance with the time slot together with the ozone removal by the ozone removal unit 14.

For example, as illustrated in FIG. 12, in a time slot when a person stays in the space 50, the voltage applied to the discharge electrode 31 is reduced with the air conditioning device 1 in operation. As a result, the ozone concentration is reduced to the extent that ozone does not adversely affect the person staying in the space. On the other hand, when no one stays in the space 50 or in a state where no one is allowed to enter the space 50, the voltage applied to the discharge electrode 31 is increased such that the ozone concentration becomes a high value. As a result, the ozone concentration is increased such that the space supplied with air is forcibly deodorized or sterilized by ozone.

The voltage applied to the discharge electrode 31 in the first mode is lower than the voltage applied to the discharge electrode 31 in the second mode operated so as to be forcibly deodorized or sterilized by ozone. However, the voltage value of the first mode is set such that dust can be efficiently collected by the electrical precipitator unit 10 even at the voltage applied to the discharge electrode 31 in the first mode. The voltage applied in the second mode is, for example, the maximum value that can be applied by the electrical precipitator unit 10. As a result, the amount of ozone generated at the electrical precipitator unit 10 can be maximized and the ozone concentration can be increased rapidly.

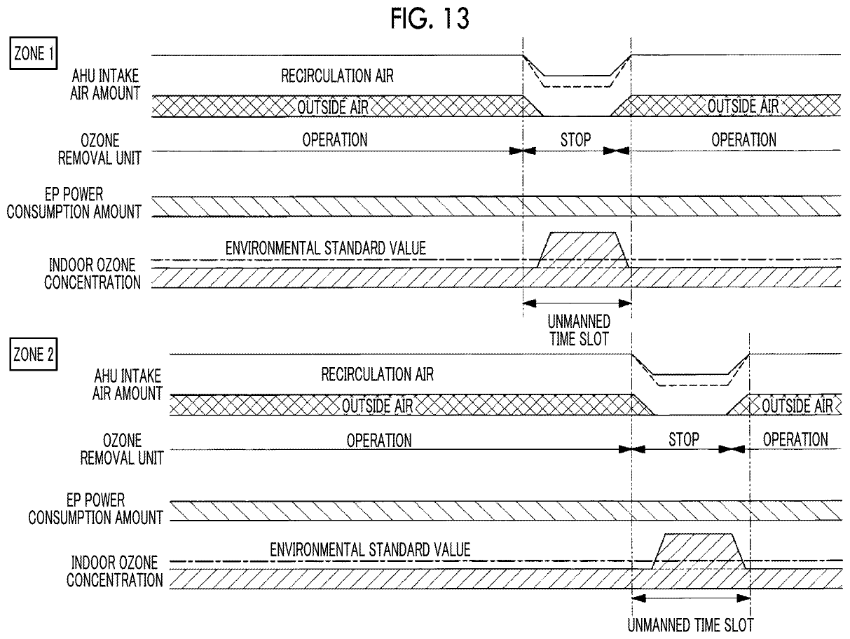

In the present embodiment, an ozone removal unit 14 is installed. Accordingly, in a case where the ozone removal unit 14 in the first mode is capable of sufficiently removing ozone such that the ozone becomes stable and equal to or less than the environmental standard value, the voltage applied to the discharge electrode 31 of the electrical precipitator unit 10 does not necessarily have to be lowered. In other words, as illustrated in FIG. 13, the voltage applied in the first mode as well as the voltage applied in the second mode may be set to the maximum value that can be applied by the electrical precipitator unit 10 and, in this case, the collection efficiency in the first mode can be enhanced.

A washing liquid supply unit 15 for washing the electrical precipitator unit 10 may be installed at the AHU 3 as illustrated in FIGS. 2 and 3. A liquid such as water, hypochlorous acid water, ozone water, and so on is supplied from the washing liquid supply unit 15 to the collecting electrode 32 via a supply pipe 16, and the liquid flows on the surface of the collecting electrode 32. A valve 17 is installed on the supply pipe 16, and the valve 17 controls the start and stop of the supply of the liquid supplied to the collecting electrode 32. As a result, it is possible to wash the dust attached on the surface of the collecting electrode 32 while sterilizing the collecting electrode 32. Hypochlorous acid water or ozone water is desirable for more effective sterilization and washing. The liquid that has flowed on the surface of the collecting electrode 32 is discharged to the outside of the AHU 3 via a drain pipe 19 as a drain. In the AHU 3 that is large or the like, the liquid that has flowed on the surface of the collecting electrode 32 may be recovered and the recovered liquid may be returned to the washing liquid supply unit 15 via a recirculation pipe 18 to be reused.

The ozone is maintained at a high concentration even in the first mode, and thus the electrical precipitator unit 10, the filter unit 12, and so on can be sterilized.

According to the air conditioning device 1 according to the present embodiment, the electrical precipitator unit 10 and the filter unit 12 are provided upstream of the air conditioning unit 13, and collection efficiency improvement can be achieved without an increase in pressure loss unlike in a case where a HEPA filter is installed. The present embodiment is particularly suitable in the case of adoption in an apparatus where it is difficult to adopt a HEPA filter and the amount of treatment air is large. By installing the electrical precipitator unit 10, dust can be collected by the electrical precipitator unit 10 and the filter unit 12 is capable of collecting the dust charged by passing through the electrical precipitator unit 10.

The inventor has confirmed that the efficiency of collection of fine particles (submicron particles), viruses, and the like hardly collectible with an existing medium-efficiency particulate air filter can be increased to at least 95% as a result. Since the pressure loss does not increase, energy consumption attributable to power can be reduced as compared with a case where a HEPA filter is installed. Here, the efficiency of collection of fine particles (submicron particles), viruses, and the like is in accordance with a mask application reference in the medical field. The collection efficiency of a mask in the medical field is set to 95% by the DOP method (0.3 μm particle) in the application reference. Although the actual collection efficiency of HEPA filter-equivalent masks is 99.97%, due to breathing difficulties, masks in the medical field have been served with the efficiency of collection of viruses and the like set to be equivalent to 95%. Even at the 95% collection efficiency confirmed in the present embodiment, practical use is possible from the viewpoint of virus removal.

The following findings have been obtained by the inventors. In other words, as for the electrical precipitator unit 10, when the input power per air amount is increased, the collection efficiency of the whole including the electrical precipitator unit 10 and the filter unit 12 is improved as illustrated in FIG. 16. This is because the amount of submicron particle charge also increases and thus the performance of the filter unit 12 as well as the electrical precipitator unit 10 is improved to increase the total efficiency. A coarse filter is preferred over the medium-efficiency particulate air filter 33 for filter washing and regeneration. An efficiency of approximately 80% or more can be achieved even in the case of a coarse filter, and the performance is significantly improved compared to the current medium-efficiency particulate air filter alone.

However, with the input power increased, the ozone concentration also increases, and thus the ozone generated at the electrical precipitator unit 10 is removed by the ozone removal unit 14 for an operation at or below the environmental standard value (0.1 ppm).

In the present embodiment, the total collection efficiency of submicron particles significantly rises, as compared with the filter unit 12 alone, as a result of the combination with the electrical precipitator unit 10. However, from the viewpoint of virus removal from the space or the like, it is desirable that the exhibited performance is equivalent to 95% or more of aerosol particles adopted in a medical mask. In order to meet this requirement, in the present embodiment, the operation and stop of the ozone removal unit 14 are switched such that it is possible to collect 95% or more of 0.3 μm particles of the same size as aerosol particles while maintaining the ozone concentration. As a result, it is possible to reduce ozone concentration and ensure aerosol particle collection efficiency at the same time.

The air conditioning device according to the present embodiment may include a fan coil unit (hereinafter, referred to as “FCU”) or the like. In other words, an FCU may be installed instead of the AHU 3 in the present embodiment. As in the case of the AHU 3, the FCU has, for example, the electrical precipitator unit 10, the control unit 11, the filter unit 12, the air conditioning unit 13, the ozone removal unit 14, and so on. Application is possible to both a newly installed FCU incorporating the electrical precipitator unit 10 and the ozone removal unit 14 and an FCU where the electrical precipitator unit 10 and the ozone removal unit 14 are additionally installed. Using an FCU capable of supplying a large amount of air, it is possible to perform air purification by deodorization or sterilization with respect to a large space. For an increase in ozone concentration, it is desirable to maximize the input power of the electrical precipitator unit 10 to maximize the amount of ozone generation and operate an FCU with the amount of treatment air reduced.

### Second Embodiment

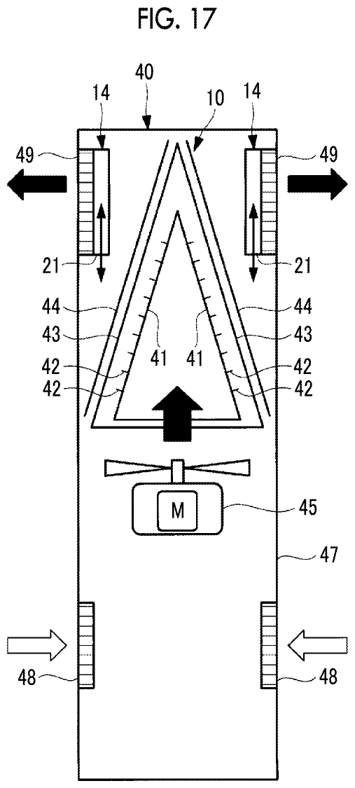

Next, an air purification device according to a second embodiment of the present disclosure will be described with reference to FIG. 17. Detailed descriptions of the configurations and actions that overlap with those of the first embodiment will be omitted.

The air purification device according to the present disclosure is not limited to the above case where the air purification device is applied to the AHU 3 and may be an air purification device without an air conditioning unit. An air purification device 40 according to the second embodiment of the present disclosure is, for example, a tower-type device as illustrated in FIG. 17. In this case, the air purification device 40 has the electrical precipitator unit 10, the control unit 11, a filter unit 44, the ozone removal unit 14, a blower 45, and so on. In a casing 47 of the air purification device 40, the electrical precipitator unit 10, a control unit, the filter unit 44, the ozone removal unit 14, the blower 45, and so on are installed.

The casing 47 is provided with a suction port 48 in the lower portion thereof and a discharge outlet 49 in the upper portion thereof. The air suctioned from the suction port 48 is sent upward from the lower part of the casing 47 by the blower 45 and is supplied to the outside from the discharge outlet 49. The blower 45 is, for example, installed below the electrical precipitator unit 10. The installation position of the blower 45 is not limited to this example and may be anywhere insofar as air is capable of flowing in the casing 47 and can be supplied to the outside.

The electrical precipitator unit 10 has a discharge electrode 41 and a collecting electrode 43. The discharge electrode 41 has a plurality of corona discharge portions 42. The corona discharge portion 42 is installed in the main body portion and is installed in a spiny shape from the main body portion toward the collecting electrode 43.

The discharge electrode 41 is a linear member and is inclined with respect to the gas flow in the inlet portion. Here, the upstream portion of the gas flow of the electrical precipitator unit 10 is positioned downward in the direction of gravity, and the downstream side of the gas flow is positioned upward in the direction of gravity. The discharge electrode 41 is installed such that two discharge electrodes 41 are combined to mutually support a load on the downstream side of the gas flow and the upstream side of the gas flow is wider than the downstream side of the gas flow.

The collecting electrode 43 has a plate-shaped member formed of a wire mesh or the like and is installed so as to face the discharge electrode 41. The plate-shaped member of the collecting electrode 43 is a conductive member having an opening portion and is, for example, a wire mesh or punching metal.

The plate-shaped member of the collecting electrode 43 is inclined with respect to the gas flow in the inlet portion. The collecting electrode 43 is installed such that two plate-shaped members are combined to mutually support a load on the downstream side of the gas flow and the upstream side of the gas flow is wider than the downstream side of the gas flow.

The collecting electrode 43 is positioned above the discharge electrode 41 and installed so as to cover the discharge electrode 41, but the discharge electrode 41 and the collecting electrode 43 are mutually separated and electrically insulated.

As illustrated in FIG. 17, the electrical precipitator unit 10 further includes the filter unit 44 installed on the side of the surface opposite to the surface where the discharge electrode 41 is provided with respect to the collecting electrode 43. The filter unit 44 is, for example, a medium-efficiency particulate air filter or a coarse filter. By further providing the filter unit 44, the collection efficiency of the electrical precipitator unit 10 as a whole can be improved. It is desirable that the filter unit 44 is finer in terms of specifications than a wire mesh. The material of the filter unit 44 is not particularly limited. The filter unit 44 is optional.

The electrical precipitator unit 10 may be wet type so that the electrical precipitator unit 10 is washed and, in this case, a liquid spray is installed inside the air purification device 40. The liquid spray is, for example, installed below the electrical precipitator unit 10. A liquid such as water, hypochlorous acid water, ozone water, and so on is sprayed from below the collecting electrode 43 to the collecting electrode 43 from the liquid spray, and the liquid flows on the surface of the collecting electrode 43. As a result, it is possible to wash the dust attached on the surface of the collecting electrode 43 while sterilizing the collecting electrode 43. Hypochlorous acid water or ozone water is desirable for more effective sterilization and washing.

The ozone removal unit 14 can be similar in configuration to the ozone removal unit 14 of the first embodiment. For example, the ozone removal unit 14 has the removal main body portion 21 described as the modification example of the first example in the first embodiment. The removal main body portion 21 has a slidable configuration. In the first mode, the removal main body portion 21 is installed such that the surface of the removal main body portion 21 is positioned at a position intersecting the flow path in the casing 47 (for example, upstream side of the discharge outlet 49). In the second mode, the removal main body portion 21 is installed such that the surface of the removal main body portion 21 is positioned away from the flow path.

[Ozone Supply Control Method]

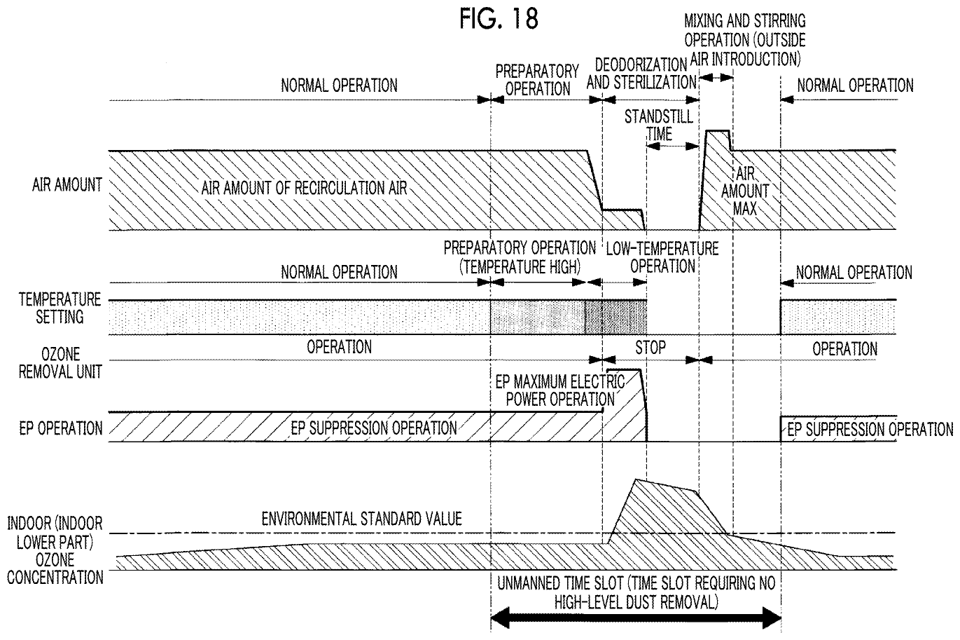

Next, with reference to FIG. 18, a control method for intensively supplying high-concentration ozone to a human action area will be described, which is applicable to both the air conditioning device according to the first embodiment of the present disclosure and the air purification device according to the second embodiment. Here, the human action area is, for example, the space of 2 m height or less from a floor surface. By deodorization or sterilization within the range of this space, adverse effects on humans can be removed or reduced, and thus the operation efficiency of the air conditioning or purification device is improved.

As described above, the temperature and air amount of the air conditioning device are controlled as follows so that a limited range from a floor surface is filled with high-concentration ozone. This control is executed by, for example, the control unit 11.

With a person staying in the space, the air conditioning device is operated normally. The electrical precipitator unit 10 is capable of removing fine particles. The ozone removal unit 14 is operated and the ozone concentration is reduced so as not to exceed the environmental standard value.

Basically, in a time slot when no one stays in the space, deodorization or sterilization work is prepared first. This is work for being capable of effectively supplying cold air to the lower part of the space in the next stage of deodorization or sterilization work. In this preparatory stage, the air amount is set to, for example, the maximum. As a result, the indoor environment is settled in a short time. The temperature of air supplied by the air conditioning device is set high and the humidity is also set high. Examples of the setting include 50% or more in relative humidity at 28° C. At this time, the electrical precipitator unit 10 is turned OFF or is operated to the same extent as in the normal operation. The humidity is raised so that sterilization with ozone is more effectively performed as it is generally known that viruses are difficult to survive in high-humidity conditions.

Next, deodorization or sterilization work is performed. The air amount is reduced in this stage. Then, the temperature of air supplied by the air conditioning device is set low. For example, the temperature is set to be approximately 3 degrees or more lower than the indoor environment. The humidity control is turned OFF. As a result, the ozone concentration is maintained. Then, the electric power of the electrical precipitator unit 10 is set to the maximum and the ozone generation amount is increased. Ozone removal by the ozone removal unit 14 is stopped. Considering effects on the human body, it is desirable to adjust the indoor ozone concentration to 0.1 ppm or more and 0.25 ppm or less. With a safety measure taken regarding the entry of people, the concentration described above can be further increased and sterilization can be effectively performed in a short time. As a result of the treatment air amount, temperature, and humidity setting and operation of the electrical precipitator unit 10, ozone concentration-raised high-density cold air is supplied to the lower part of the space. At this time, mixing and stirring with the indoor air are suppressed, filling with the cold air occurs slowly and gradually from the place near the floor surface, and the cold air is introduced into the entire lower part of the space. Then, the part can be intensively deodorized or sterilized by allowing the high-ozone concentration air to stand still at the lower part of the space.

In the example of the air conditioning device described above, the air temperature is lowered by the heat exchanger in order to fill the human action area with ozone more effectively. However, in addition, fine water mist may be sprayed into the air to be blown and the air temperature may be lowered using the temperature drop attributable to vaporization of the mist.

In a case where the deodorization or sterilization work is completed, an operation for reducing the ozone concentration in the space is performed. For example, outside air is introduced into the space. The electrical precipitator unit 10 is turned OFF or operated to the same extent as in the normal operation while increasing the air amount of the air conditioning device. The ozone removal unit 14 is operated and ozone is removed by the ozone removal unit 14. The ozone concentration is reduced by actively mixing and stirring the indoor air. A person can stay in the space after a state where the ozone concentration has reliably decreased is confirmed. Although normal operation initiation immediately after the end of an unmanned time slot is illustrated in FIG. 18, the normal operation initiation may precede the end of the unmanned time slot.

Although the above description with reference to FIG. 18 describes an example of the air conditioning device in which the ozone removal unit 14 is installed, the present disclosure is not limited to this example. In other words, the control for intensive high-concentration ozone supply to the human action area may be performed by combining an air purification device equipped with an ozone removal unit 14 and an air conditioning device without an ozone removal unit.

The generated ozone may be adsorbed onto the medium-efficiency particulate air filter 33. In order to desorb the adsorbed ozone from the medium-efficiency particulate air filter 33, a period in which the operation of the electrical precipitator unit 10 is turned OFF is provided. For example, ozone desorption is performed by operating only a fan while turning OFF the electrical precipitator unit 10 in an unmanned state or in a time slot when a high level of dust removal is not required. In a manned normal operation time slot of the air conditioning device, ozone generated at the electrical precipitator unit 10 is adsorbed on the filter, and thus the ozone concentration in the space gradually rises, and the ozone concentration becomes constant at ozone adsorption saturation.

### Third Embodiment

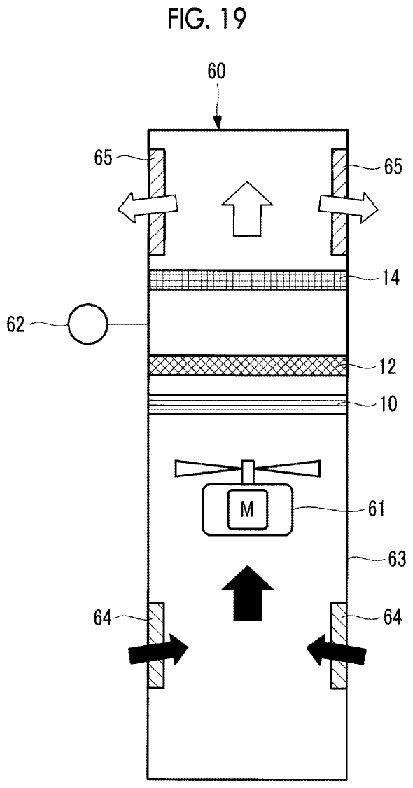

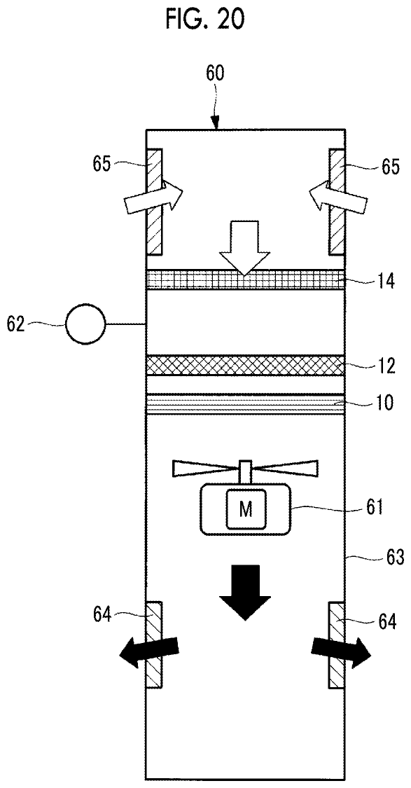

Next, an air purification device according to a third embodiment of the present disclosure will be described with reference to FIGS. 19 and 20. Detailed descriptions of the configurations and actions that overlap with those of the first and second embodiments will be omitted.

An air purification device 60 according to the present embodiment is, for example, a tower-type device as illustrated in FIGS. 19 and 20. In this case, the air purification device 60 has the electrical precipitator unit 10, a control unit, the filter unit 12, the ozone removal unit 14, a blower 61, an ozone concentration measuring unit 62, and so on. In a casing 63 of the air purification device 60, the electrical precipitator unit 10, a control unit, the filter unit 12, the ozone removal unit 14, the blower 61, and so on are installed. In the flow path inside the casing 63, the electrical precipitator unit 10, the filter unit 12, and the ozone removal unit 14 are installed in this order upward from below.

The casing 63 is provided with a lower opening portion 64 in the lower portion thereof and an upper opening portion 65 in the upper portion thereof.

The electrical precipitator unit 10 is fixedly installed in the casing 63. The electrical precipitator unit 10 has the discharge electrode 31 and the collecting electrode 32 as illustrated in FIGS. 7 to 9 and as in the first embodiment. The electrical precipitator unit 10 having the discharge electrode 41 and the collecting electrode 43 may be installed in the air purification device 60 as illustrated in FIG. 17 and as in the second embodiment. In this case, the electrical precipitator unit 10 may be wet type so that the electrical precipitator unit 10 is washed and, in this case, a liquid spray is installed inside the air purification device 60. The liquid spray may be, for example, installed below the electrical precipitator unit 10, between the electrical precipitator unit 10 and the filter unit 12, or on the downstream side of the filter unit 12 in the case of washing together with the filter unit 12.

The filter unit 12 is fixedly installed in the casing 63. The filter unit 12 is equipped with the medium-efficiency particulate air filter 33 normally used in the AHU 3. The medium-efficiency particulate air filter 33 may be a filter with a pre-charged filter material. In the present embodiment, the filter unit 12 is optional. The filter unit 12 that has a coarse filter instead of the medium-efficiency particulate air filter may be installed. The presence or absence of the filter unit 12 and the type of the filter are appropriately selected in accordance with particulate matter removal performance.

The ozone removal unit 14 is in the form of a filter carrying an ozone decomposition catalyst and is fixedly installed in the casing 63. Air always passes through the ozone removal unit 14.

The blower 61 is, for example, installed below the electrical precipitator unit 10. The installation position of the blower 61 is not limited to this example and may be anywhere insofar as air is capable of flowing in the casing 63 and can be supplied to the outside. The blower 61 is controlled by the control unit and is capable of sending air upward from below or downward from above in the casing 63 by changing the direction of rotation.

The blower 61 is, for example, an axial blower such as a propeller fan and is capable of forward rotation and reverse rotation. The forward rotation of the fan of the blower 61 sends air in the forward direction, and the reverse rotation of the fan of the blower 61 sends air in the direction opposite to the forward direction. The present invention is not limited to this example, and the fan may rotate only in one direction and be incapable of forward rotation and reverse rotation. In this case, a forward fan and a reverse fan may be provided and the fan to be operated may be switched.

The control unit controls the blower 61 and switches between an air purification mode (hereinafter, also referred to as “first mode”) and an ozone sterilization mode (hereinafter, also referred to as “second mode”). In the first mode (air purification mode), as illustrated in FIG. 19, the air suctioned from the lower opening portion 64 positioned on the vertical lower side of the air purification device 60 is sent upward from the lower part of the casing 63 by the blower 61 and is supplied to the outside from an upper opening portion 65 positioned on the vertical upper side of the air purification device 60. As a result, air is sent such that the ozone removal unit 14 is positioned on the downstream side of the electrical precipitator unit 10. As a result, in the first mode, ozone-removed air is supplied to the outside from the downstream portion of the flow path, that is, the upper opening portion 65, and thus no ozone is supplied to the space 50 with which the air that has passed through the electrical precipitator unit 10.

In the first mode, air passes through the electrical precipitator unit 10 and the filter unit 12 in this order. Accordingly, at the electrical precipitator unit 10, particulate matter is collected and a sufficient amount of charge can be applied to the particulate matter, and thus a strong electrostatic force acts on the main body of the filter unit 12. As a result, the collection efficiency of the filter unit 12, fine particle collection efficiency in particular, is significantly improved. Accordingly, high dust removal performance is exhibited in the first mode.

In the second mode (ozone sterilization mode), as illustrated in FIG. 20, the air suctioned from the upper opening portion 65 is sent downward from above the casing 63 by the blower 61 and is supplied to the outside from the lower opening portion 64. As a result, air is sent such that the ozone removal unit 14 is positioned on the upstream side of the electrical precipitator unit 10. As a result, ozone generated by the corona discharge at the discharge electrode 31, 41 does not pass through the ozone removal unit 14 and flows toward the lower opening portion 64. Accordingly, in the second mode, ozone-containing air is supplied to the outside from the downstream portion of the flow path, that is, the lower opening portion 64, and thus ozone generated by corona discharge at the discharge electrode 31, 41 is supplied to the space 50 supplied with the air that has passed through the electrical precipitator unit 10.

In the second mode, the air blowing amount is smaller than in the first mode, and thus it is possible to generate high-concentration ozone and supply ozone-containing air to the space 50. The blower 61 has a smaller amount of air in the reverse blowing direction than in the forward blowing direction, and thus the blower 61 may be installed such that the supply direction in the first mode is the forward direction of the blower 61. In this manner, by applying the forward rotation side with a large air amount to the first mode (air purification mode) and applying the reverse rotation side with a relatively small air amount to the second mode (ozone sterilization mode), high-concentration ozone is supplied in the second mode with a small air amount.

In the second mode in which ozone-containing air is supplied, air is supplied to the space 50 from the lower portion of the air purification device 60. Accordingly, ozone denser than air slowly accumulates from the lower portion to the upper portion of the space 50, and the ozone concentration in the space 50 can be increased upward from below. Accordingly, it is possible to intensively sterilize a range in which a person touches a thing, that is, a place close to a floor surface.

Next, the control of the air purification device 60 according to the present embodiment will be described.

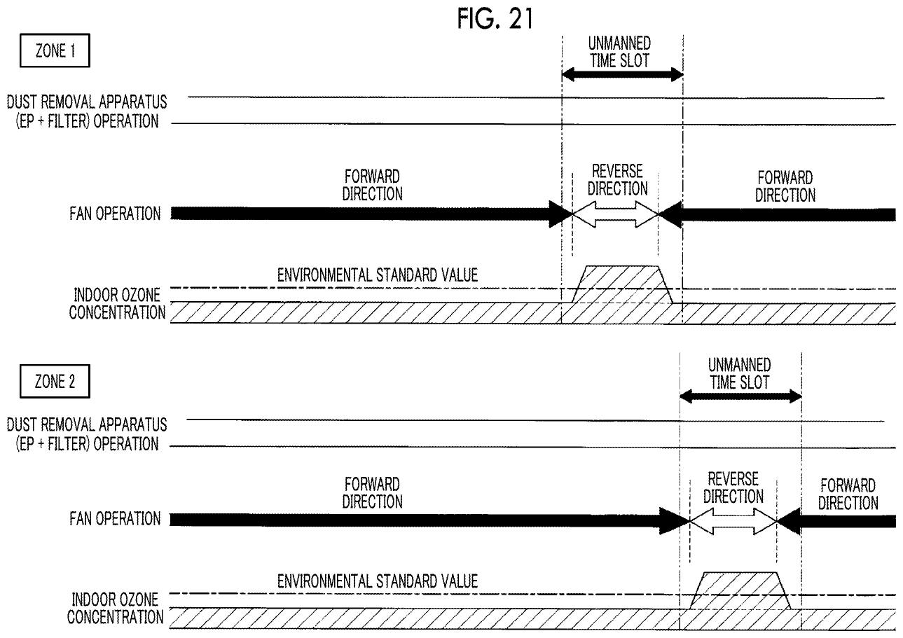

For example, as illustrated in FIG. 21, in a time slot when a person stays in the space 50, a switch to the first mode is performed with the air purification device 60 in operation and ozone-removed air supply is started such that the ozone concentration becomes equal to or less than the environmental standard value. As a result, the ozone concentration is reduced to the extent that ozone does not adversely affect the person staying in the space. On the other hand, when no one stays in the space 50 or in a state where no one is allowed to enter the space 50, a switch to the second mode is performed and ozone-containing air supply is started such that the ozone concentration becomes a high value. As a result, the ozone concentration is increased such that the space supplied with air is forcibly deodorized or sterilized by ozone.

A predetermined CT value may be ensurable so that the effect of sterilization or deodorization in the space 50 is obtained. The CT value is a value (ppm·min) expressed by the product of the ozone concentration (ppm) and the time (min) of contact with an object to be treated at the ozone concentration. Accordingly, even in a case where the ozone concentration is low, by setting a long contact time, it is possible to ensure a CT value equivalent to that in the case of contact with a high ozone concentration in a short time. For example, the second mode may be set to a relatively long time in a case where the absolute ozone concentration is suppressed or in a case where there is a limit to the amount of ozone generated at the electrical precipitator unit 10.

For example, the ozone concentration measuring unit 62 is installed in the space 50 supplied with the air that has passed through the electrical precipitator unit 10 and the filter unit 12 from the air purification device 60. The ozone concentration measuring unit 62 measures the ozone concentration in the space. Data related to the measured ozone concentration is transmitted from the ozone concentration measuring unit 62 to the control unit. The control unit receives a signal related to the measurement data from the ozone concentration measuring unit 62. After a switch to the second mode, the control unit determines whether or not a predetermined CT value has been exceeded based on the measured ozone concentration and the time of measurement of the ozone concentration. The control unit switches from the second mode to the first mode when it is determined that the predetermined CT value has been exceeded and continues the second mode when it is determined that the predetermined CT value is not exceeded.

The ozone concentration measuring unit 62 may be installed in the casing 63 so as to be capable of measuring the ozone concentration in the space. By installing the ozone concentration measuring unit 62 at a predetermined height, the CT value at a position lower than the ozone concentration measuring unit 62 can be reliably ensured. The ozone concentration measuring unit 62 may be provided in the casing 63 such that the height position can be changed.

In an office building or a large space where people cannot enter depending on the time slot (for example, a theater), it is also possible to automatically switch between the first mode and the second mode using an unmanned time slot at night.

In the case of returning from a state where the ozone concentration in the space 50 is high to a state where the ozone concentration is low and satisfies the environmental standard value, a switch is performed from the second mode of high concentration to the first mode of low concentration. At this time, the operation of the electrical precipitator unit 10 may be stopped in order to efficiently reduce the ozone concentration. As a result, the ozone removal unit 14 acts only to remove the ozone in the space. In a case where the ozone removal unit 14 is of high removal capacity, the operation of the electrical precipitator unit 10 does not necessarily have to be stopped.

In a case where a plurality of the air purification devices 60 are installed and the air purification devices 60 supply air to different spaces 50, as illustrated in FIG. 21, a switch to the second mode is performed for each space 50 and a target space is deodorized or sterilized. For example, in a case where deodorization or sterilization is performed in one space 50 (Zone 1 in the example illustrated in FIG. 21), only the target space is set to the second mode and the other space 50 (Zone 2 in the example illustrated in FIG. 21) remains in the first mode.

In the first mode in which ozone-removed air is supplied, air is suctioned from the lower portion of the air purification device 60. Accordingly, when the mode is switched from the second mode to the first mode, the ozone-containing air accumulated in the lower portion of the space 50 can be taken in, and the ozone removal unit 14 is capable of reliably removing the ozone. In the second mode in which ozone-containing air is supplied, air is supplied to the space 50 from the lower portion of the air purification device 60. Accordingly, high-density ozone slowly accumulates from the lower portion to the upper portion of the space 50, and the ozone concentration in the space 50 can be increased upward from below. Accordingly, high-concentration ozone can be intensively supplied to the human action area. Here, the human action area is, for example, the space of 2 m height or less from a floor surface. By deodorization or sterilization within the range of this space, adverse effects on humans can be removed or reduced, and thus the operation efficiency of the air purification device is improved.

The air purification device and the air conditioning device described in each embodiment described above are, for example, grasped as follows.

An air purification device according to the present disclosure includes: a flow path through which air flows; an electrical precipitator unit (10) installed in the flow path including a discharge electrode (31, 41) having a main body portion (31A) and a corona discharge portion (31B, 31C, 42) for corona discharge protruding from the main body portion and a collecting electrode (32, 43) installed to face the discharge electrode; an ozone removal unit (14) installed in the flow path and capable of removing ozone contained in the flowing air; and a first control unit (11) switching between a first mode in which air from which the ozone is removed is supplied to an outside from a downstream portion of the flow path and a second mode in which air containing the ozone is supplied to the outside from the downstream portion of the flow path.

According to this configuration, the electrical precipitator unit installed in the flow path through which the air flows includes the discharge electrode and the collecting electrode, corona discharge occurs by voltage application to the discharge electrode, and dust (particulate matter) charged as a result of the corona discharge is collected on the collecting electrode.

Although ozone is generated by corona discharge at the discharge electrode, the ozone removal unit is capable of removing ozone contained in the flowing air. The first control unit switches between the first mode and the second mode. In the first mode, the ozone-removed air is supplied to the outside from the downstream portion of the flow path, and no ozone is supplied to the space supplied with the air that has passed through the electrical precipitator unit. In the second mode, the ozone-containing air is supplied to the outside from the downstream portion of the flow path, and thus ozone generated by corona discharge at the discharge electrode is supplied to the space supplied with the air that has passed through the electrical precipitator unit.

In the air purification device according to the present disclosure, the ozone removal unit may be installed on a downstream side of the electrical precipitator unit, and the first control unit may control the ozone removal unit to cause the ozone removal unit to remove the ozone in the first mode and to stop removal of the ozone by the ozone removal unit in the second mode.

According to this configuration, the ozone removal unit installed downstream of the electrical precipitator unit is capable of removing ozone contained in the flowing air. The first control unit controls the ozone removal unit to switch between the first mode and the second mode. In the first mode, ozone is removed by the ozone removal unit and the ozone-removed air is supplied to the outside from the downstream portion of the flow path, and no ozone is supplied to the space supplied with the air that has passed through the electrical precipitator unit. In the second mode, ozone removal by the ozone removal unit is stopped and the ozone-containing air is supplied to the outside from the downstream portion of the flow path, and thus ozone generated by corona discharge at the discharge electrode is supplied to the space supplied with the air that has passed through the electrical precipitator unit.

In the air purification device according to the present disclosure, the ozone removal unit may include a filter-shaped removal main body portion (21) carrying an ozone decomposition catalyst, and a drive unit (22) driving the removal main body portion, and the drive unit may be controlled by the first control unit to drive the removal main body portion and switch between the first mode and the second mode, the removal main body portion may be changed in position or direction such that air that has passed through the electrical precipitator unit and the filter unit is positioned or directed to pass through the removal main body portion in the first mode, and the removal main body portion may be changed in position or direction such that air that has passed through the electrical precipitator unit and the filter unit is positioned or directed not to pass through the removal main body portion in the second mode.

According to this configuration, the ozone removal unit has the filter-shaped removal main body portion and the drive unit, and the removal main body portion carries an ozone decomposition catalyst and removes ozone from the air that has passed. The drive unit drives the removal main body portion to switch between the first mode and the second mode. The position or direction of the removal main body portion is changed by the drive unit. In the first mode, the removal main body portion is in a position or direction in which the air that has passed through the electrical precipitator unit passes through the removal main body portion. In the second mode, the removal main body portion is in a position or direction in which the air that has passed through the electrical precipitator unit does not pass through the removal main body portion.

In the air purification device according to the present disclosure, the removal main body portion may be supported so as to be rotatable about a shaft installed on the removal main body portion, and the drive unit may drive the removal main body portion to rotate the removal main body portion.

According to this configuration, the removal main body portion is rotated by the drive unit about the shaft installed in the removal main body portion in a supported state. By changing the direction of the removal main body portion to intersect the flow path, the removal main body portion has a direction in which the air that has passed through the electrical precipitator unit passes through the removal main body portion. By changing the direction of the removal main body portion to be parallel to the flow path, the removal main body portion has a direction in which the air that has passed through the electrical precipitator unit does not pass through the removal main body portion.

In the air purification device according to the present disclosure, the removal main body portion may be supported so as to be movable in a direction parallel to a plane direction of the removal main body portion, and the drive unit may drive the removal main body portion to move the removal main body portion.

According to this configuration, the removal main body portion is moved by the drive unit in the direction parallel to the plane direction of the removal main body portion. By the removal main body portion being moved to and installed at a position intersecting the flow path, the removal main body portion has a position where the air that has passed through the electrical precipitator unit passes through the removal main body portion. By the removal main body portion being moved to and installed at a position away from the position intersecting the flow path, the removal main body portion has a position where the air that has passed through the electrical precipitator unit does not pass through the removal main body portion.

In the air purification device according to the present disclosure, the ozone removal unit may include a filter-shaped removal main body portion (26) carrying an ozone decomposition catalyst, a damper (27) as a plate-shaped member changing a flow direction of the air, and a drive unit (28) driving the damper, and the drive unit may be controlled by the first control unit to drive the damper and switch between the first mode and the second mode, the damper may be changed in position or direction such that air that has passed through the electrical precipitator unit is positioned or directed to pass through the removal main body portion in the first mode, and the damper may be changed in position or direction such that air that has passed through the electrical precipitator unit is positioned or directed not to pass through the removal main body portion in the second mode.

According to this configuration, the ozone removal unit is in the form of a filter carrying an ozone decomposition catalyst and removes ozone from the air that has passed. The damper is a plate-shaped member and changes the flow direction of the air. The damper is driven by the drive unit to switch between the first mode and the second mode. The position or direction of the damper is changed by the drive unit. In the first mode, the damper is in a position or direction in which the air that has passed through the electrical precipitator unit passes through the removal main body portion. In the second mode, the removal main body portion is in a position or direction in which the air that has passed through the electrical precipitator unit does not pass through the removal main body portion.

In the air purification device according to the present disclosure, the ozone removal unit may be an ultraviolet lamp (24) emitting an ultraviolet ray capable of decomposing the ozone, and the ultraviolet lamp may be controlled by the first control unit to switch between the first mode and the second mode, the ultraviolet lamp may irradiate air that has passed through the electrical precipitator unit with the ultraviolet ray in the first mode, and the ultraviolet lamp may stop irradiating air that has passed through the electrical precipitator unit with the ultraviolet ray in the second mode.

According to this configuration, the ozone removal unit is an ultraviolet lamp emitting ultraviolet rays, ozone is decomposed by the ultraviolet rays, and ozone is removed from the air that has passed. The first control unit controls the ultraviolet lamp to switch between the first mode and the second mode. In the first mode, the ultraviolet lamp irradiates the air that has passed through the electrical precipitator unit with ultraviolet rays. In the second mode, the ultraviolet lamp stops irradiating the air that has passed through the electrical precipitator unit with ultraviolet rays.

The air purification device according to the present disclosure may further include a blowing unit (61) sending air in the flow path from one side to the other side, the ozone removal unit may be filter-shaped and carry an ozone decomposition catalyst, and the first control unit may control the blowing unit, the air may be sent such that the ozone removal unit is positioned on a downstream side of the electrical precipitator unit and the air from which the ozone is removed is supplied to the outside from the downstream portion of the flow path in the first mode, and the air may be sent such that the ozone removal unit is positioned on an upstream side of the electrical precipitator unit and the air containing the ozone is supplied to the outside from the downstream portion of the flow path in the second mode.

According to this configuration, the air in the flow path is sent from one side to the other side by the blowing unit. The first control unit controls the blowing unit to switch between the first mode and the second mode. In the first mode, the air is sent such that the ozone removal unit is positioned on the downstream side of the electrical precipitator unit. As a result, in the first mode, ozone-removed air is supplied to the outside from the downstream portion of the flow path, and thus no ozone is supplied to the space supplied with the air that has passed through the electrical precipitator unit. In the second mode, the air is sent such that the ozone removal unit is positioned on the upstream side of the electrical precipitator unit. As a result, ozone generated by the corona discharge at the discharge electrode does not pass through the ozone removal unit and flows toward the downstream portion of the flow path. As a result, in the second mode, ozone-containing air is supplied to the outside from the downstream portion of the flow path, and thus ozone generated by corona discharge at the discharge electrode is supplied to the space supplied with the air that has passed through the electrical precipitator unit.

The air purification device according to the present disclosure may further include an ozone concentration measuring unit installed in a space supplied with air that has passed through the electrical precipitator unit and measuring ozone concentration in the space, and the first control unit may switch between the first mode and the second mode based on the measured ozone concentration.

According to this configuration, the ozone concentration measuring unit installed in the space supplied with the air that has passed through the electrical precipitator unit measures the ozone concentration in the space and switches between the first mode and the second mode based on the measured ozone concentration. For example, the first mode and the second mode are switched based on a CT value calculated by ozone concentration-contact time multiplication.

In the air purification device according to the present disclosure, the collecting electrode may be a plate-shaped member and have a plate surface provided parallel to a gas flow direction, and the corona discharge portion may include a first corona discharge portion (31B) protruding from the main body portion toward an upstream side in the gas flow direction in one side end portion of the main body portion and a second corona discharge portion (31C) protruding from the main body portion toward a downstream side in the gas flow direction in the other side end portion of the main body portion.

According to this configuration, the plate surface of the collecting electrode, which is a plate-shaped member, is provided parallel to the gas flow direction, and gas flows between the discharge electrode and the collecting electrode. The first corona discharge portion protrudes from the main body portion toward the upstream side in the gas flow direction in one side end portion of the main body portion of the discharge electrode, and the second corona discharge portion protrudes from the main body portion toward the downstream side in the gas flow direction in the other side end portion of the main body portion of the discharge electrode. The discharge electrode is capable of causing ionic wind to flow by causing corona discharge from the corona discharge portion toward the collecting electrode. Since the plurality of stages of corona discharge portions are provided, the collection performance is improved. Further, since the electrical precipitator unit is provided with the plurality of stages of corona discharge portions, a sufficient amount of charge can be applied to particles and a strong electrostatic force acts on the medium-efficiency particulate air filter unit, and thus the collection performance is improved.

In the air purification device according to the present disclosure, a negative energization may be applied to the discharge electrode.

According to this configuration, a negative energization is applied to the discharge electrode, stable discharge becomes possible, and ozone is likely to be generated during discharge.

The air purification device according to the present disclosure may further include a second control unit (11) adjusting a voltage or energization condition applied to the discharge electrode of the electrical precipitator unit.

According to this configuration, the voltage or energization condition applied to the discharge electrode of the electrical precipitator unit is adjusted by the second control unit. As a result, the amount of ozone generated by corona discharge at the discharge electrode is adjusted, and thus the ozone concentration in the space can be increased or decreased.

The air purification device according to the present disclosure may further include a filter unit (12, 44) installed in the flow path and including a medium-efficiency particulate air filter or a coarse filter.

According to this configuration, dust in gas is collected by the filter unit installed in the air flow path. The filter unit having a medium-efficiency particulate air filter or a coarse filter is capable of reducing pressure loss and replacement frequency.

An air conditioning device (1) according to the present disclosure includes: the air purification device described above; and an air conditioning unit (13), in which the air conditioning unit supplies air relatively high in temperature to a space and then supplies air relatively small in air amount and relatively low in temperature to the space, and the first control unit stops removal of the ozone by the ozone removal unit.

According to this configuration, air relatively high in temperature is supplied to the space and then air relatively small in air amount and relatively low in temperature is supplied to the space. At this time, the ozone removal by the ozone removal unit is stopped and the ozone concentration is increased. As a result, mixing and stirring with indoor air is suppressed, filling with cold air gradually occurs from near the floor surface, and the air is introduced into the entire lower space. Then, the lower portion of the space is intensively deodorized or sterilized.

### REFERENCE SIGNS LIST

1: Air conditioning device
2: External air conditioner
4, 5, 6: Duct
7, 8: Damper
9: Casing
10: Electrical precipitator unit
11: Control unit
12: Filter unit
13: Air conditioning unit
14: Ozone removal unit
15: Washing liquid supply unit
16: Supply pipe
17: Valve
18: Recirculation pipe
19: Drain pipe
21: Removal main body portion
22: Drive unit
23: Rotary shaft
24: Ultraviolet lamp
25: Blower
26: Removal main body portion
27: Damper
28: Drive unit
29: Rotary shaft
31: Discharge electrode
31A: Main body portion
31B, 31C: Corona discharge portion
32: Collecting electrode
33: Medium-efficiency particulate air filter
40: Air purification device
41: Discharge electrode
42: Corona discharge portion
43: Collecting electrode
44: Filter unit
45: Blower
47: Casing
48: Suction port
49: Discharge outlet
50, 50A, 50B, 50C: Space
60: Air purification device
61: Blower
62: Ozone concentration measuring unit
63: Casing
64: Lower opening portion
65: Upper opening portion

1. An air purification device comprising:
a flow path through which air flows;
an electrical precipitator unit including a discharge electrode having a main body portion and a corona discharge portion for corona discharge protruding from the main body portion and a collecting electrode installed to face the discharge electrode and installed in the flow path;
an ozone removal unit installed in the flow path and capable of removing ozone contained in the flowing air; and
a first control unit switching between a first mode in which air from which the ozone is removed is supplied to an outside from a downstream portion of the flow path and a second mode in which air containing the ozone is supplied to the outside from the downstream portion of the flow path.

2. The air purification device according to claim 1, wherein
the ozone removal unit is installed on a downstream side of the electrical precipitator unit, and
the first control unit controls the ozone removal unit to cause the ozone removal unit to remove the ozone in the first mode and to stop removal of the ozone by the ozone removal unit in the second mode.

3. The air purification device according to claim 2, wherein
the ozone removal unit includes
a filter-shaped removal main body portion carrying an ozone decomposition catalyst, and
a drive unit driving the removal main body portion, and
the drive unit is controlled by the first control unit to drive the removal main body portion and switch between the first mode and the second mode, the removal main body portion is changed in position or direction such that air that has passed through the electrical precipitator unit is positioned or directed to pass through the removal main body portion in the first mode, and the removal main body portion is changed in position or direction such that air that has passed through the electrical precipitator unit is positioned or directed not to pass through the removal main body portion in the second mode.

4. The air purification device according to claim 3, wherein
the removal main body portion is supported so as to be rotatable about a shaft installed on the removal main body portion, and
the drive unit drives the removal main body portion to rotate the removal main body portion.

5. The air purification device according to claim 3, wherein
the removal main body portion is supported so as to be movable in a direction parallel to a plane direction of the removal main body portion, and
the drive unit drives the removal main body portion to move the removal main body portion.

6. The air purification device according to claim 2, wherein
the ozone removal unit includes
a filter-shaped removal main body portion carrying an ozone decomposition catalyst,
a damper as a plate-shaped member changing a flow direction of the air, and
a drive unit driving the damper, and
the drive unit is controlled by the first control unit to drive the damper and switch between the first mode and the second mode, the damper is changed in position or direction such that air that has passed through the electrical precipitator unit is positioned or directed to pass through the removal main body portion in the first mode, and the damper is changed in position or direction such that air that has passed through the electrical precipitator unit is positioned or directed not to pass through the removal main body portion in the second mode.

7. The air purification device according to claim 1, wherein
the ozone removal unit is an ultraviolet lamp emitting an ultraviolet ray capable of decomposing the ozone, and
the ultraviolet lamp is controlled by the first control unit to switch between the first mode and the second mode, the ultraviolet lamp irradiates air that has passed through the electrical precipitator unit with the ultraviolet ray in the first mode, and the ultraviolet lamp stops irradiating air that has passed through the electrical precipitator unit with the ultraviolet ray in the second mode.

8. The air purification device according to claim 1, further comprising a blowing unit sending air in the flow path from one side to the other side, wherein
the ozone removal unit is filter-shaped and carries an ozone decomposition catalyst, and
the first control unit controls the blowing unit, the air is sent such that the ozone removal unit is positioned on a downstream side of the electrical precipitator unit and the air from which the ozone is removed is supplied to the outside from the downstream portion of the flow path in the first mode, and the air is sent such that the ozone removal unit is positioned on an upstream side of the electrical precipitator unit and the air containing the ozone is supplied to the outside from the downstream portion of the flow path in the second mode.

9. The air purification device according to claim 1, further comprising an ozone concentration measuring unit installed in a space supplied with air that has passed through the electrical precipitator unit and measuring ozone concentration in the space, wherein
the first control unit switches between the first mode and the second mode based on the measured ozone concentration.

10. The air purification device according to claim 1, wherein
the collecting electrode is a plate-shaped member and has a plate surface provided parallel to a gas flow direction, and
the corona discharge portion includes a first corona discharge portion protruding from the main body portion toward an upstream side in the gas flow direction in one side end portion of the main body portion and a second corona discharge portion protruding from the main body portion toward a downstream side in the gas flow direction in the other side end portion of the main body portion.

11. The air purification device according to claim 1, wherein
a negative energization is applied to the discharge electrode.

12. The air purification device according to claim 1, further comprising a second control unit adjusting a voltage or energization condition applied to the discharge electrode of the electrical precipitator unit.

13. The air purification device according to claim 1, further comprising a filter unit installed in the flow path and including a medium-efficiency particulate air filter or a coarse filter.

14. An air conditioning device comprising:
the air purification device according to claim 1; and
an air conditioning unit, wherein
the air conditioning unit supplies air relatively high in temperature to a space and then supplies air relatively small in air amount and relatively low in temperature to the space, and
the first control unit stops removal of the ozone by the ozone removal unit.
